(19) United States
(12) Patent Application Publication
(10) Pub. No.: US 2020/0392203 A1
(43) Pub. Date: Dec. 17, 2020

(54) IMMUNOTHERAPY AGAINST TRANSFERRIN RECEPTOR 1 (TFR1)-TROPIC ARENAVIRUSES

(71) Applicant: Yeda Research and Development Co. Ltd., Rehovot (IL)

(72) Inventors: Ron DISKIN, Rehovot (IL); Hadas COHEN-DVASHI, Rehovot (IL)

(73) Assignee: Yeda Research and Development Co. Ltd., Rehovot (IL)

(21) Appl. No.: 16/638,816

(22) PCT Filed: Aug. 14, 2018
(86) PCT No.: PCT/IL2018/050901

§ 371 Date: Feb. 13, 2020

(30) Foreign Application Priority Data
Aug. 14, 2017 (IL) 253984

(51) Int. Cl. C07K 14/705; G01N 33/569; A61P 31/14; A61K 38/00

(52) U.S. Cl. CPC C07K 14/70582; G01N 33/56983; G01N 2333/08; A61K 38/00; C07K 2319/30; A61P 31/14

(57) ABSTRACT

A composition of matter comprising an isolated soluble polypeptide comprising an amino acid sequence of a Transferrin receptor protein 1 (TfR1) apical domain is disclosed, the soluble polypeptide being capable of binding an Arenavirus. A fusion protein comprising an amino acid sequence of a TfR1 apical domain and an amino acid sequence of IgG Fc, the fusion protein capable of binding an Arenavirus, is also disclosed.

### FIELD AND BACKGROUND OF THE INVENTION

The present invention, in some embodiments thereof, relates to soluble fragments of Transferrin receptor protein 1 (TfR1) apical domain and, more particularly, but not exclusively, to the use of same for the treatment or prevention of an Arenavirus viral infection.

Viral hemorrhagic fevers are a major global health problem. The recent Ebola crisis demonstrated how fast epidemics could spread with modern transportation and emphasized the importance of having effective countermeasures before the onset of such deadly outbreaks. Effective immunotherapy holds a great promise against deadly viruses.

‘New World’ (NW) Arenaviruses are zoonotic enveloped, single-stranded RNA viruses, prevalent in the South and North Americas, and are classified into four different clades. They are carried by rodent-reservoirs and cause acute illness upon infecting humans, often with hemorrhagic-fever manifestations. Pathogenic NW Arenaviruses include the clade-B Machupo (MACV), Junin (JUNV), Guanarito (GTOV), and Sabia (SBAV) viruses that infect people in Bolivia, Argentina, Venezuela, and Brazil, respectively. In addition, the North American clade-A/B Whitewater Arroyo virus (WWAV) or some of its genetically close isolates may also be pathogenic to humans. All of these viruses utilize TfR1 as their entry receptor [Radoshitzky et al., Nature (2007) 446: 92-96] and the ability to utilize the human-TfR1 (hTfR1) distinguishes them from the non-pathogenic members.

Arenaviruses have a trimeric class-I glycoprotein with a GP1 subunit that adopts a unique fold and mediates receptor recognition. It was demonstrated that neutralizing monoclonal antibodies against JUNV target the receptor-binding site on GP1, but no cross-neutralization of other NW Arenaviruses was observed using these antibodies or using sera from JUNV-convalescent patients [Mahmutovic et al., Cell Host Microbe (2015) 18: 705-713] due to structural variations of the receptor binding sites [Mahmutovic et al., (2015) supra; Brouillette et al, J Virol (2017) 91]. These antibodies can rescue animals from lethal challenges with JUNV [Zeitlin et al., Proc Natl Acad Sci USA (2016) 113: 4458-4463].

Targeting the apical domain of TfR1 using antibodies was previously suggested as a therapeutic approach [Abraham et al. Nat Struct Mol Biol (2010) 17: 438-444]. Helguera et al. identified an anti-hTfR1 antibody, ch 128.1, which efficiently inhibited entry mediated by the glycoproteins of five Arenaviruses, as well as replication of infectious Junin virus [Helguera et al., J Virol. (2012) 86(7): 4024-8]. According to Helguera et al., all NW hemorrhagic fever Arenaviruses utilize a common hTfR1 apical-domain epitope and therapeutic agents targeting this epitope, including ch 128.1, can be broadly effective in treating South American hemorrhagic fevers.

Aptamers that target hTfR1 apical-domain were also developed and suggested for inhibiting NW hemorrhagic fever Mammarenavirus entry [Maier et al., Mol Ther Nucleic Acids (2016) 5: e321].

U.S. Pat. No. 9,439,973 and U.S. Patent Application No. 2015/125516 provide isolated ribonucleic acid aptamers of 60 bases or less which bind a human transferrin receptor and inhibit a New World Arenavirus from infecting a cell, but do not compete with human transferrin for binding to the human transferrin receptor.

### SUMMARY OF THE INVENTION

According to an aspect of some embodiments of the present invention there is provided a composition of matter comprising an isolated soluble polypeptide comprising an amino acid sequence of a Transferrin receptor protein 1 (TfR1) apical domain, the soluble polypeptide being capable of binding an Arenavirus.

According to an aspect of some embodiments of the present invention there is provided a composition of matter comprising a soluble polypeptide comprising an amino acid sequence of a TfR1 apical domain as set forth in SEQ ID NO: 6, the soluble polypeptide being capable of binding an Arenavirus.

According to an aspect of some embodiments of the present invention there is provided a fusion protein comprising an amino acid sequence of a TfR1 apical domain and an amino acid sequence of IgG Fc, the fusion protein capable of binding an Arenavirus.

According to an aspect of some embodiments of the present invention there is provided a pharmaceutical composition comprising the composition of matter or fusion protein of some embodiments of the invention, and a pharmaceutically acceptable carrier.

According to an aspect of some embodiments of the present invention there is provided a method of treating or preventing an Arenavirus viral infection or disease associated therewith in a subject in need thereof, the method comprising administering to the subject a therapeutically effective amount of the composition of matter or fusion protein of some embodiments of the invention, thereby treating or preventing the Arenavirus viral infection or disease associated therewith in the subject. According to an aspect of some embodiments of the present invention there is provided an isolated polynucleotide encoding the polypeptide or fusion protein of some embodiments of the invention.

According to an aspect of some embodiments of the present invention there is provided a nucleic acid construct comprising the isolated polynucleotide of some embodiments of the invention.

According to an aspect of some embodiments of the present invention there is provided a method of producing a polypeptide, the method comprising introducing the nucleic acid construct of some embodiments of the invention into a host cell; and culturing the host cell under conditions suitable for expressing the polypeptide.

According to an aspect of some embodiments of the present invention there is provided a method of diagnosing an Arenavirus viral infection in a subject, the method comprising: (a) contacting a biological sample from the subject with the composition of matter or fusion protein of some embodiments of the invention, under conditions which allow the formation of immunocomplexes between an Arenavirus and the soluble polypeptide or the fusion protein; and (b) determining a level of the immunocomplexes in the biological sample, wherein an increase in level of the immunocomplexes beyond a predetermined threshold with respect to a level of the immunocomplexes in a biological sample from a healthy individual is indicative of the Arenavirus viral infection. According to some embodiments of the invention, the amino acid sequence is devoid of the long loop.

According to some embodiments of the invention, the amino acid sequence comprises at least one deletion, insertion or point mutation that renders the TfR1 soluble.

According to some embodiments of the invention, the at least one point mutation comprises a substitution of a hydrophobic residue with a hydrophilic residue.

According to some embodiments of the invention, the point mutation is at an interface between the apical domain and the protease-like domain of the TfR1.

According to some embodiments of the invention, the at least one point mutation abolishes a glycosylation site of the TfR1.

According to some embodiments of the invention, the glycosylation site comprises an N—X—S glycosylation motif.

According to some embodiments of the invention, the Serine of the N—X—S glycosylation motif is mutated to any amino acid or mimetic thereof with the proviso that the amino acid is not Threonine.

According to some embodiments of the invention, the Serine of the N—X—S glycosylation motif is mutated to Alanine or mimetic thereof.

According to some embodiments of the invention, the Asparagine of the N—X—S glycosylation motif is mutated to any amino acid or mimetic thereof with the proviso that the amino acid not Asparagine.

According to some embodiments of the invention, the polypeptide comprises a stabilizing moiety.

According to some embodiments of the invention, the stabilizing moiety comprises a cysteine residue.

According to some embodiments of the invention, the cysteine residue comprises at least one cysteine residue at N- and/or C-termini of the polypeptide.

According to some embodiments of the invention, the polypeptide is of a length not exceeding 180 amino acid residues.

According to some embodiments of the invention, the TfR1 is of a human, a rodent, or a bat origin.

According to some embodiments of the invention, the rodent is a White-throated woodrat.

According to some embodiments of the invention, the amino acid sequence of the TfR1 is as set forth in SEQ ID NO: 2, 4, 16 or 18.

According to some embodiments of the invention, the polypeptide is attached to a heterologous moiety.

According to some embodiments of the invention, the heterologous moiety is capable of inducing an antibody dependent cellular-mediated cytotoxicity (ADCC) response.

According to some embodiments of the invention, the heterologous moiety is for increasing avidity of the polypeptide.

According to some embodiments of the invention, the heterologous moiety is for multimerization.

According to some embodiments of the invention, the heterologous moiety is a proteinaceous moiety.

According to some embodiments of the invention, the proteinaceous moiety is selected from the group consisting of an immunoglobulin, a galactosidase, a glucuronidase, a glutathione-S-transferase (GST), a carboxy terminal peptide (CTP) from chorionic gonadotrophin (CGβ), and a chloramphenicol acetyltransferase (CAT).

According to some embodiments of the invention, the proteinaceous moiety is an immunoglobulin.

According to some embodiments of the invention, the immunoglobulin is an IgG Fc.

According to some embodiments of the invention, the composition of matter is as set forth in SEQ ID NO: 8.

According to some embodiments of the invention, the composition of matter is as set forth in SEQ ID NO: 23.

According to some embodiments of the invention, the heterologous moiety is a non-proteinaceous moiety.

According to some embodiments of the invention, the non-proteinaceous moiety is selected from the group consisting of polyethylene glycol (PEG), Polyvinyl pyrrolidone (PVP), poly(styrene comaleic anhydride) (SMA), and divinyl ether and maleic anhydride copolymer (DIVEMA).

According to some embodiments of the invention, the fusion protein is as set forth in SEQ ID NO: 8.

According to some embodiments of the invention, the fusion protein is as set forth in SEQ ID NO: 23.

According to some embodiments of the invention, the composition of matter or fusion protein of some embodiments of the invention is capable of neutralizing the Arenavirus.

According to some embodiments of the invention, the composition of matter or fusion protein of some embodiments of the invention is capable of initiating antibody-dependent cellular cytotoxicity (ADCC).

According to some embodiments of the invention, the composition of matter or fusion protein of some embodiments of the invention is for use in treating or preventing an Arenavirus viral infection or disease associated therewith in a subject in need thereof.

According to some embodiments of the invention, the disease is a hemorrhagic fever.

According to some embodiments of the invention, the Arenavirus is selected from the group consisting of, Junin (JUNV), Machupo (MACV), Guanarito (GTOV), Sabia (SABV), Whitewater Arroyo (WWAV), Chapare (CHPV), Cupixi (CPXV), Tacaribe (TCRV), Bear Canyon (BCNV), Tamiami (TAMV), Big Brushy Tank (BBTV), Catarina (CTNV), Skinner Tank (SKTV), and Tonto Creek (TTCV).

According to some embodiments of the invention, the nucleic acid sequence is as set forth in SEQ ID NO: 1, 3, 15 or 17.

According to some embodiments of the invention, the nucleic acid sequence is as set forth in SEQ ID NO: 5.

According to some embodiments of the invention, the nucleic acid sequence is as set forth in SEQ ID NO: 7.

According to some embodiments of the invention, the nucleic acid sequence is as set forth in SEQ ID NO: 22.

According to some embodiments of the invention, the nucleic acid construct of some embodiments of the invention further comprises a signal peptide.

According to some embodiments of the invention, the method further comprises recovering the polypeptide.

According to some embodiments of the invention, the method further comprises corroborating the diagnosis using a diagnostic assay selected from antigen level measurement, antibody level measurement, virus isolation and/or genomic detection by reverse transcriptase-polymerase chain reaction (RT-PCR).

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D illustrate the design of a soluble apical domain from TfR1. FIG. 1A—Overview of the TfR1/GP1 complex structure (PDB ID: 3KAS). Two GP1 molecules from MACV (grey) bound to the dimeric human-TfR1 (light-blue and green). FIG. 1B—The apical domain of TfR1 (orange) is imbedded within in the protease-like domain (light blue), and together with the helical dimerization domain (magenta) makes one complete copy of the TfR1 molecule. FIG. 1C—Sequence alignment of human TfR1 (GenBank: AB209254.1/UniProtKB-P02786) and Neotoma Albigula (NA) TfR1 (GenBank KF982058/UniProt A0A060BIS8) and soluble apical domain (sAD). The numbering scheme follows the human-TfR1 numbering and the sequence of the human TfR1 is colored according to the color scheme as in ‘FIG. 1B’ & ‘FIG. 1D’. The potential N-linked glycosylation sites are indicated with black arrows. FIG. 1D-A close-up view of the hydrophobic interface of the apical domain (orange) and the protease-like domain (light blue). The hydrophobic residues that were mutated in sAD are shown in green.

FIGS. 2A-E illustrate that the designed apical domain makes a soluble and stable protein that effectively binds a range of GP1 domains. FIG. 2A—Size exclusion chromatography profile of the soluble apical domain after affinity purification demonstrates a predominant monodisperse monomeric peak (mark with an asterisk). FIG. 2B—Representative circular dichroism spectrum of the sAD demonstrates a well-folded protein. FIG. 2C—Melt experiment of sAD. Circular dichroism signal was monitored at wavelength of 222 nm. The sAD was stable until 55° C. (light-blue shaded region), with an estimated T_{M} of approximately 65° C. (red line). FIG. 2D—A spider graph showing the dissociation constants (K_{D}) between sAD and the indicated GP1 domains from clades B & A/B mammarenaviruses, as measured using SPR. FIG. 2E—Crystal structure of sAD in complex with GP1_{MACV}. The GP1 domain is shown using surface representation (white) and sAD is presented as ribbon diagram in rainbow colors from the N′-terminus (blue) to the C′-terminus (red). N-linked glycans are shown using sticks, as well as Tyr211 of sAD.

FIGS. 3A-C illustrate that Arenacept is biologically active against the pathogenic viruses. FIG. 3A—Confocal fluorescence imaging of HEK293 cells, transiently transfected with genes encoding GPCs from the indicated viruses and stained with Arenacept. Nuclei were stained with DAPI (blue), membranes were stained with wheat germ agglutinin (green) and Arenacept was visualized using fluorescent anti-human Ab (red). Scale bars represent 20 μm. FIG. 3B—Neutralization of pseudotyped viruses. Graphs show representative neutralization of viruses that bear the spike complexes from the indicated viruses. Infection was monitored in a stable HEK293 cell line that overly-expresses hTfR1 using a luciferase reporter gene. Error bars show standard deviations from technical replicates. The reported IC₅₀ values are means of at least three independent experiments. FIG. 3C—antibody dependent cellular-mediated cytotoxicity (ADCC) assay.

FIG. 4 illustrates that the sAD adopts the same overall structure as the apical domain of hTfR1. A ribbon diagram showing the apical domain of hTfR1 (PDB ID: 3KAS) in orange superimposed on sAD that is shown in blue. The right view is 90° rotated in respect to the view on the left. Tyr211 that is a central residue at the interface with GP1 is shown. Residues 301-326 of hTfR1 that were omitted in sAD are colored pink.

FIG. 5 illustrates that the asymmetric unit contains four copies of the sAD/GP1_{MACV} complex. The eight chains that make the asymmetric unit are shown using a unique color for each chain. The right view is 90° rotated in respect to the view on the left. The chains are rendered using tubes for which the radii are proportional to the B-factor. There are differences between the protein pairs in the asymmetric unit; some are well defined in the electron density, having low B-factors (e.g. green/cyan), others are less defined and hence have higher B-factor (e.g. purple/orange).

FIGS. 6A-B illustrate that a dimeric Arenacept has higher potency compared with monomeric sAD. Neutralization assay of pseudotyped viruses bearing the spike complex of MACV and JUNV by Arenacept (black) and sAD (blue), showing elevated potency due to dimerization and indicating the effect of avidity. Since the MW of sAD and Arenacept significantly differ, the neutralization data is compared using a molarity scale. Without the effect of avidity, sAD can neutralize MACV to some degree but was practically inert toward JUNV at the range of concentrations used for this assay. These observations agree with the measured K_{D} values for sAD with JUNV and MACV (i.e. 1 μM and 4 nM, respectively).

FIGS. 7A-B illustrate conformational changes of sAD in respect to the native apical domain. FIG. 7A—A ribbon diagram showing the structure of sAD/GP1_{MACV} complex, in blue and white respectively that is superimposed on hTfR1/GP1_{MACV} complex (PDB ID: 3KAS), which are colored orange and gray, respectively. The long loop that connects strands 011-6 and 011-7 is changing position in sAD compared to hTfR1 and is highlights in green (sAD). This loop in hTfR1 originally includes residues 301-326 (pink) that were eliminated from sAD. FIG. 7B—The negatively charged Glu294 of hTfR1 is forming a salt-bridge with Lys169 from GP1. In the case of sAD, Glu340 from αII-2 that replaces an alanine residue of hTfR1 is projecting to the same direction as Glu294 of hTfR1. This Glu340 of sAD forms a similar salt-bridge with Lys169 of GP1_{MACV}.

FIGS. 8A-E illustrate measurements of K_{D} values between sAD and GP1 s from TfR1-tropic viruses. GP1-Fc fusion proteins were immobilized on a protein-A coated SPR sensor chip and sAD was injected in a series of increasing concentrations (i.e. 5, 50, 250, 500, 1000 nM) using a single cycle kinetic scheme. Representative blank-subtracted sensorgrams are shown in orange and a 1:1 binding model that was fitted to the data is shown in black. Below each sensorgram a residual plot is showing the quality of the fitted model. The calculated K_{D} values are shown for each GP1. Each binding experiment was repeated twice.

FIG. 9 illustrates that mutating Tyr211 reduces the potency of Arenacept. A neutralization assay of pseudotyped virus bearing the spike complex of JUNV by Arenacept (blue) and an Y211A-Arenacept (black) indicating loss of potency.

FIG. 10 illustrates a sequence alignment of human (i.e. Homo Sapiens) TfR1 as set forth in SEQ ID NO: 2, White-throated woodrat (i.e. Neotoma Albigula) TfR1 as set forth in SEQ ID NO: 4, Jamaican fruit bat (i.e. Artibeus Jamaicensis) TfR1 as set forth in SEQ ID NO: 12, and Hispid cotton rat (i.e. Sigmodon Hispidus) TfR1 as set forth in SEQ ID NO: 21. Green illustrates the missing residues in each sequence. Blue illustrates the long loop residues. Magenta illustrates Tyr211.

FIGS. 11A-D illustrate neutralization of pseudoviruses bearing the spike complexes of the indicated viruses. The neutralization of Arenacept (black curves) is compared to neutralization of the N206A variant of Arenacept (red curves).

### DESCRIPTION OF SPECIFIC EMBODIMENTS OF THE INVENTION

The present invention, in some embodiments thereof, relates to soluble fragments of Transferrin receptor protein 1 (TfR1) apical domain and, more particularly, but not exclusively, to the use of same for the treatment or prevention of an Arenavirus viral infection.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting. Rodent born Arenaviruses can cause severe life threatening hemorrhagic fevers when infecting humans. It is highly desired to have effective countermeasures against these viruses. Due to their efficient transmission they pose a severe risk for outbreaks and might be exploited as bioterrorism weapons. Ideally, one would want to have a single remedy that will be effective against many or even all the pathogenic strains in this family. However, despite the fact that all pathogenic New World (NW) Arenaviruses utilize transferrin receptor 1 (TfR1) as a cellular receptor, their viral glycoproteins are highly diversified, impeding efforts for isolating cross-neutralizing antibodies.

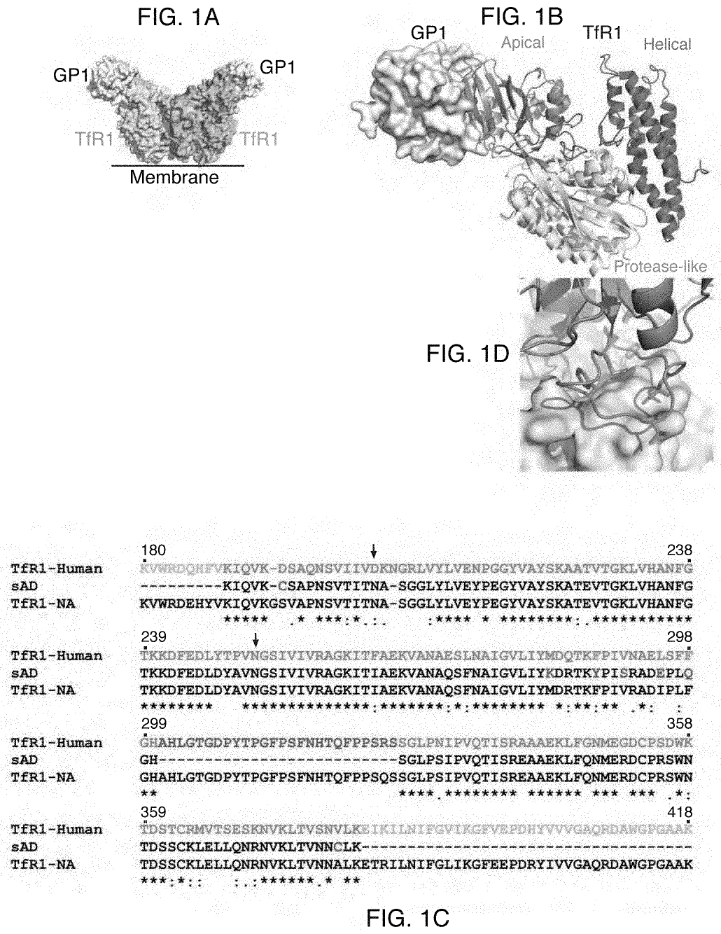

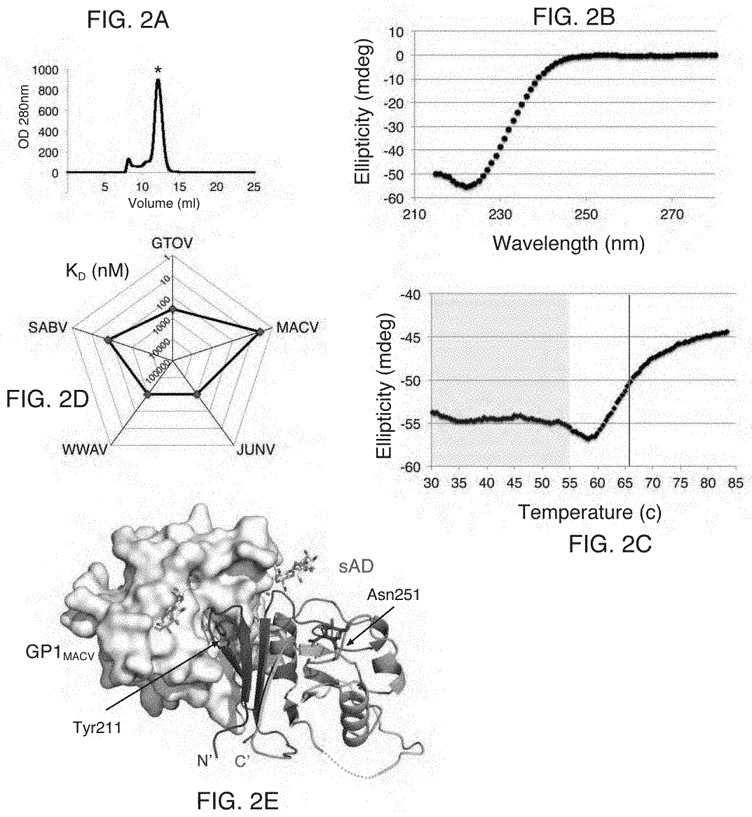

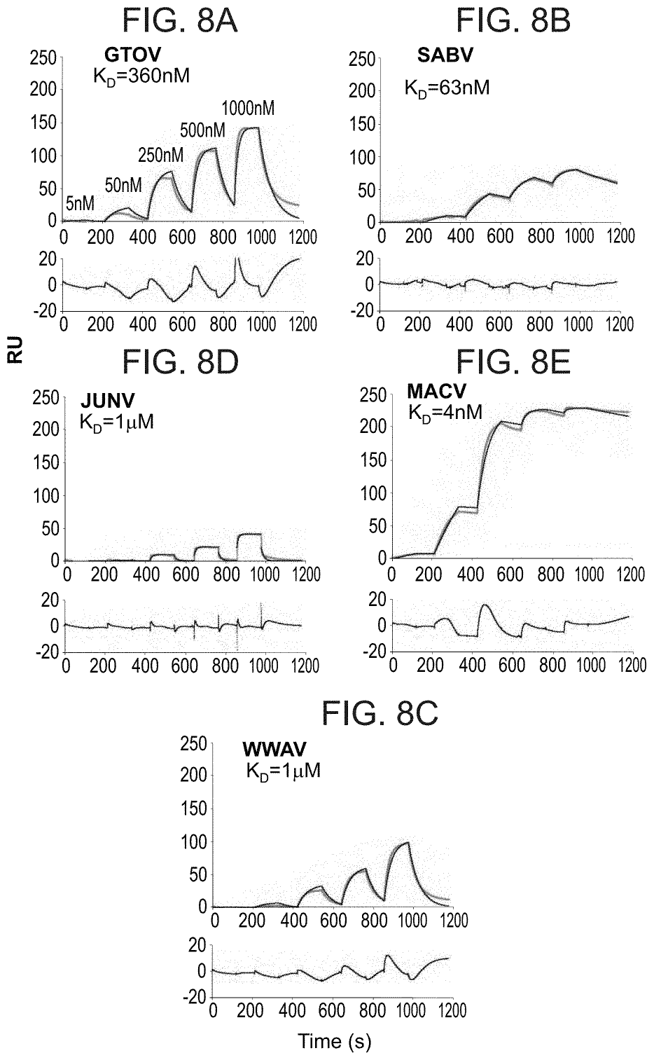

While reducing the present invention to practice, the present inventors have designed and generated a TfR1-mimicry protein that blocks the Arenavirus's GP1 receptor binding site and thereby prevents viral infection. Accordingly, a soluble apical domain (sAD) was designed as an isolated protein for making a TfR1 receptor binding site competitor. The apical domain of TfR1 has no known biological functions and hence makes a potentially safe reagent to be injected to patients as a decoy. The design of sAD was based on the TfR1 gene from Neotoma Albigula (White-throated woodrat) in which the long loop (residues 301-326) has been removed, several hydrophobic residues that make part of the interface between the apical and the protease-like domains have been mutated, and two cysteine residues were introduced at both termini of the peptide (FIG. 1C). sAD was illustrated to be a soluble, folded and thermo-stable protein (FIGS. 2A-C), an advantageous property that would be instrumental for the ability to distribute it in regions with poor clinical and logistical infrastructures. Furthermore, sAD comprised a broad-spectrum of reactivity against GP1s from clade-B and A/B NW Arenaviruses, e.g. JUNV, MACV, GTOV, SABV and WWAV (FIGS. 8A-E).

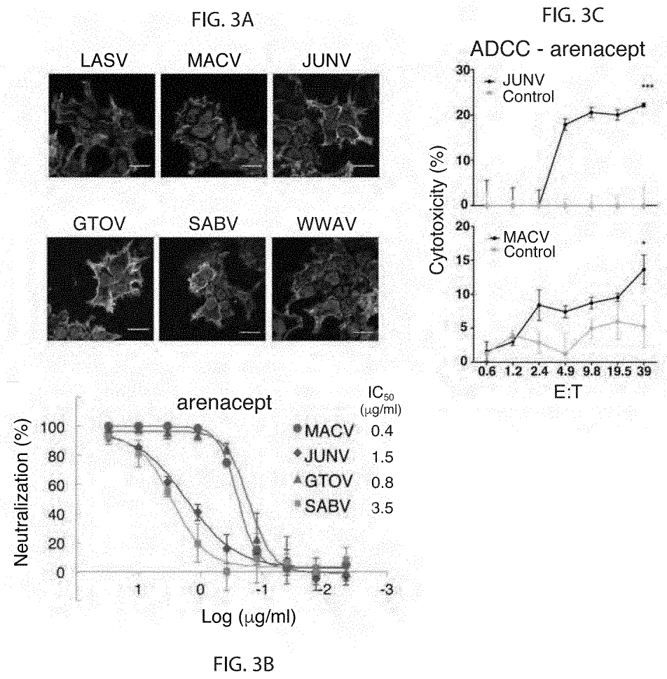

The present inventors have further constructed the sAD as an immunoadhesin by fusing to its C-terminus an Fc portion of IgG1 in a configuration that enables avidity (termed “Arenacept”). Arenacept specifically recognizes the native spike complexes of MACV, JUNV, GTOV, SABV and WWAV (FIG. 3A) and was capable of effectively neutralizing them (FIG. 3B). Arenacept was further proven as being efficient in inducing antibody-dependent cellular cytotoxicity (ADCC, FIG. 3C). Thus, beside direct neutralization of viruses, Arenacept is capable to inducing ADCC.

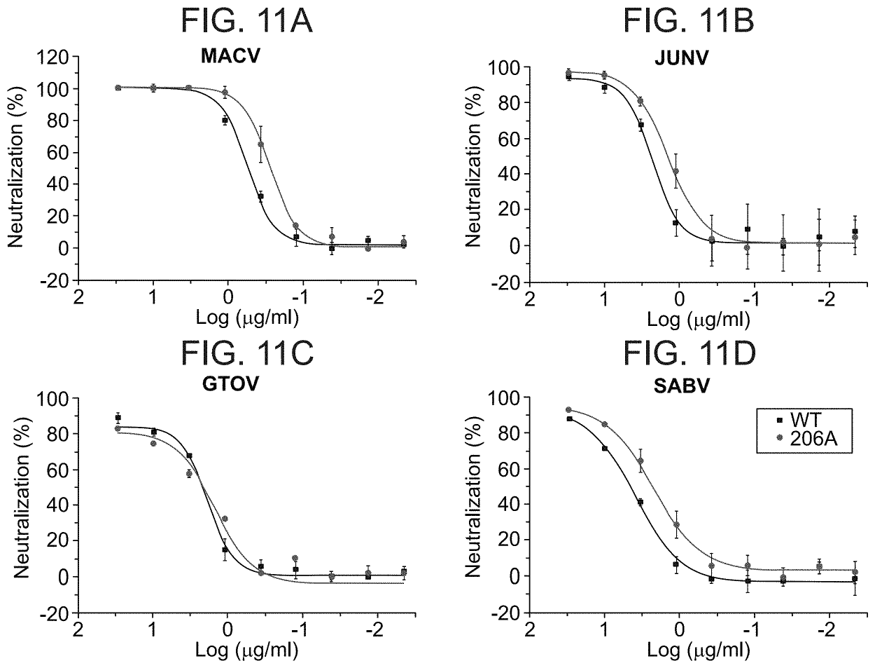

The present inventors have added further modifications to Arenacept. Specifically, Arenacept was modified to replace serine of the N—X—S glycosylation motif with alanine. It was illustrated that Arenacept^{S206A} neutralizes pseudotyped TfR1-tropic arenaviruses (FIGS. 11A-D and Table 4, hereinbelow).

Taken together, the development of Arenacept offers a promising new immunotherapeutic approach for combating infections by the notorious pathogenic NW Arenaviruses, which pose a health threat for millions of people in the endemic regions and so far had very limited options for treatment. Moreover, Arenacept can be useful for diagnosis of infection by TfR1-tropic viruses using, for example, virus overlay protein binding assay (VOPBA).

Thus, according to one aspect of the present invention there is provided a composition of matter comprising an isolated soluble polypeptide comprising an amino acid sequence of a Transferrin receptor protein 1 (TfR1) apical domain, the soluble polypeptide being capable of binding an Arenavirus.

As used herein the term “Arenavirus” refers to RNA-containing viruses that belong to the Arenaviridae family of viruses.

According to one embodiment, the Arenaviruses comprise the New World (NW) arenaviruses, i.e. the single-stranded RNA viruses, prevalent in the South and North Americas, which typically cause acute illness in humans often with hemorrhagic-fever manifestations. Arenaviruses infect host cells via GP1, which is part of trimeric envelope glycoprotein complex i.e. GP1/GP2/stable signal peptide (SSP).

The terms “spike complex” or “trimeric class 1 viral glycoprotein complex” or “trimeric envelope glycoprotein complex” as used herein all refer to the viral protein complex composed of three copies of each of the attachment glycoprotein GP1, the membrane-anchored fusion protein GP2, and the stable signal peptide (SSP). The spike complex recognizes the cellular receptors and mediates membrane fusion and host infectivity.

The term “GP1” or “Glycoprotein 1” as used herein refers to the Arenavirus envelope glycoprotein i.e. the receptor binding domain of the spike complex that mediates receptor recognition (e.g. TfR1) for entry into the host cell.

According to one embodiment, the NW arenaviruses include those of clades A, B, C, and recombinant A/B clade.

Exemplary Arenaviruses include, but are not limited to, Junin (JUNV), Machupo (MACV), Guanarito (GTOV), Sabia (SABV), Whitewater Arroyo (WWAV), Chapare (CHPV), Cupixi (CPXV), Tacaribe (TCRV), Bear Canyon (BCNV), Tamiami (TAMV), Big Brushy Tank (BBTV), Catarina (CTNV), Skinner Tank (SKTV), Tonto Creek (TTCV), Amapari virus (AMAV), Oliveros virus (OLIV) and Sabia (SBAV).

The term “Transferrin receptor protein 1” or “TfR1” as used herein refers to the cell surface receptor, also known as CD71 or P90.

According to one embodiment, the TfR1 is a mammalian TfR1.

According to one embodiment, the TfR1 is a human TfR1 or an ortholog thereof.

Exemplary human TfR1 are set forth in Accession Nos. NP 003225.2, NP 001121620.1, NP_001300894.1 or NP_001300895.1. Exemplary TfR1 orthologs include, but are not limited to, the mouse TfR1 e.g. as set forth in Accession No. NP_035768.1; the rat TfR1 e.g. as set forth in Accession No. NP_073203.1; the bat TfR1 e.g. as set forth in Accession Nos. XP_008153714.1, XP_014314708.1, XP_006092878.1, XP_014400772.1; the hamster TfR1 e.g. as set forth in Accession No. NP_001233748.1; the cat TfR1 e.g. as set forth in Accession No. NP_001009312.1; the dog TfR1 e.g. as set forth in Accession No. NP_001003111.1; the pig TfR1 e.g. as set forth in Accession No. NP_999166.1; the horse TfR1 e.g. as set forth in Accession No. NP_001075382.1; the frog TfR1 e.g. as set forth in Accession No. XP_017949664.1; and the chicken TfR1 e.g. as set forth in Accession No. NP_990587.2.

According to one embodiment, the TfR1 is a human TfR1 e.g. as set forth in SEQ ID NO: 2.

According to one embodiment, the TfR1 is of a rodent origin. Exemplary rodents include, but are not limited to, mice, rats, squirrels, prairie dogs, porcupines, beavers, guinea pigs, hamsters, gerbils, and rabbits.

According to one embodiment, the TfR1 is a woodrat TfR1. According to a specific embodiment, the TfR1 is a White-throated woodrat TfR1 (e.g. Neotoma Albigula TfR1) e.g. as set forth in SEQ ID NO: 4.

According to one embodiment, the TfR1 is a Hispid cotton rat TfR1. According to a specific embodiment, the TfR1 is a Hispid cotton rat TfR1 (e.g. Sigmodon Hispidus TfR1) e.g. as set forth in SEQ ID NO: 21.

According to one embodiment, the TfR1 is a mouse TfR1 e.g. as set forth in SEQ ID Nos: 10 or 14.

According to one embodiment, the TfR1 is a bat TfR1. According to a specific embodiment, the TfR1 is a Jamaican fruit bat TfR1 (e.g. Artibeus Jamaicensis TfR1) e.g. as set forth in SEQ ID NO: 12.

TfR1 comprises three subdomains: a “protease-like domain”, an “apical domain”, and a “helical domain”. Transferrin typically interacts with the “protease-like domain” and “helical domain” while the “apical domain” is the principal site of interaction with Arenaviral (i.e. the New World Arenavirus) glycoproteins.

As used herein “corresponds” or “corresponding” refers to an amino acid or a stretch of amino acids that is homologous in structure and/or orientation in the context of the polypeptide i.e., TfR1.

According to a one embodiment, the amino acid sequence of the “protease-like domain” of TfR1 corresponds to residues 120-608 of SEQ ID NO: 2.

According to a one embodiment, the amino acid sequence of the “apical domain” of TfR1 corresponds to residues 189-300 of SEQ ID NO: 2. Thus, it will be understood that the apical domain of TfR1 is embedded within the protease-like domain of TfR1.

According to a one embodiment, the amino acid sequence of the “helical domain” of TfR1 corresponds to residues 609-756 of SEQ ID NO: 2.

The term “corresponds to residues” refers to the position of an amino acid in an amino acid sequence in a given organism (e.g. human). Determination of the corresponding residues in other organisms (e.g. rodent, bat, etc.) can be carried out using any sequence alignment methods known to one of skill in the art.

Thus, for example, determination of the apical domain of a TfR1 (e.g. TfR1 orthologs e.g. human, mouse, rat etc.) can be carried out using any method known in the art, such as by sequence alignment software, such as the BLAST software available from the NCBI server (wwwdotncbidotnlmdotnihdotgov/BLAST/).

Generally, the present application relates to the sequence of human TfR1, e.g. as set forth in SEQ ID NO: 2, therefore “corresponds to residues” relates to the position of amino acid residues in the sequence of the human TfR1.

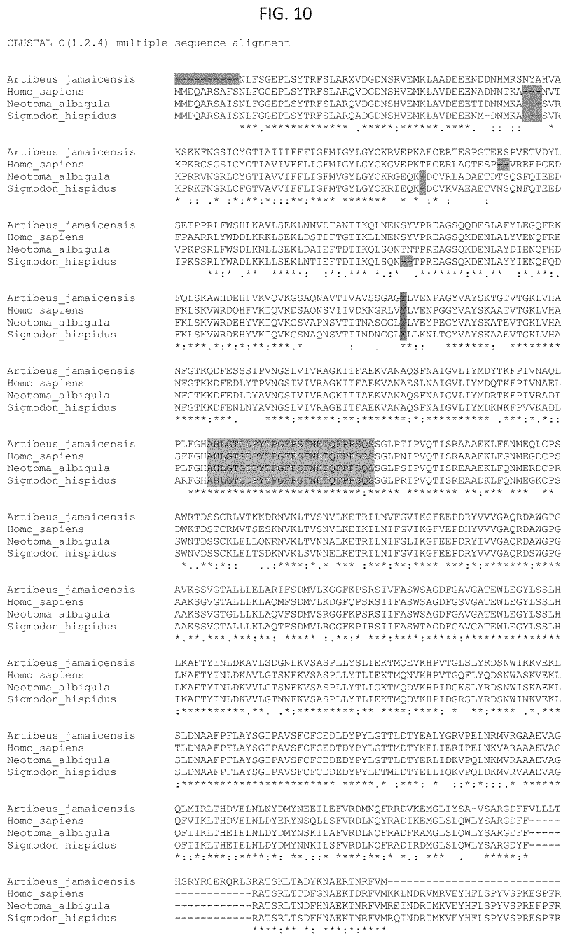

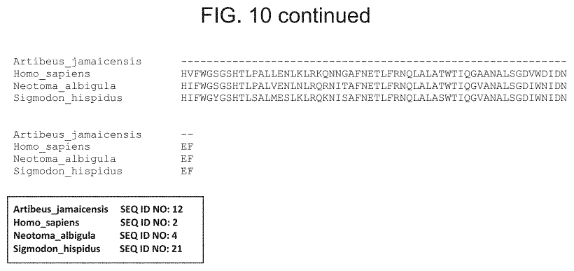

According to one embodiment, the sequence numbering of White-throated woodrat TfR1 apical domain is +1 as compared to human TfR1 (as illustrated in the sequence alignment of FIG. 10).

Thus, the isolated polypeptide of the invention comprises at least a fragment of the apical domain (e.g. at least about 20%, 30%, 40%, 50%, 60%, 70%, 80%, 90% or 100% of the 195 amino acid sequence of the apical domain) and is capable of binding an Arenavirus (e.g. the Arenavirus GP1 glycoprotein). The isolated polypeptide of the invention may further comprise fragments of the helical domain or protease-like domain (i.e. amino acid sequences of the helical domain or protease-like domain), as long as the polypeptide is soluble, isolated and capable of binding an Arenavirus.

According to one embodiment of the invention, the amino acid sequence of the isolated polypeptide comprises an amino acid sequence having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence homology or identity to the TfR1 apical domain as long as the polypeptide is soluble, isolated and capable of binding an Arenavirus.

Homology (e.g., percent homology, identity+similarity) can be determined using any homology comparison software, including for example, the BlastP or TBLASTN software of the National Center of Biotechnology Information (NCBI) such as by using default parameters, when starting from a polypeptide sequence; or the tBLASTX algorithm (available via the NCBI) such as by using default parameters, which compares the six-frame conceptual translation products of a nucleotide query sequence (both strands) against a protein sequence database.

For example, default parameters for tBLASTX include: Max target sequences: 100; Expected threshold: 10; Word size: 3; Max matches in a query range: 0; Scoring parameters: Matrix-BLOSUM62; filters and masking: Filter—low complexity regions.

The term “binding an Arenavirus” as used herein refers to the capability of at least about 50%, 60%, 70%, 80%, 90% or 100% of the polypeptides in the composition to bind the trimeric spike complex or its GP1 domain from an Arenavirus, as compared to the binding of a native TfR1 to an Arenavirus. Measuring the binding of the isolated polypeptide to an Arenavirus can be carried out using any method known in the art, such as for example, by Surface Plasmon Resonance Assay, Enzyme-linked immunosorbent (ELISA) assay, Microscale thermophoresis (MST), Bio-Layer Interferometry (BLI), Isothermal titration calorimetry (ITC), Analytical Ultracentrifugation.

According to one embodiment, binding of the isolated polypeptide to the trimeric spike complex or its GP1 domain from an Arenavirus is characterized by a K_{D} lower than 50 μM.

It should be noted that the affinity can be quantified using known methods such as, Surface Plasmon Resonance (SPR) (described in Scarano S, Mascini M, Turner A P, Minunni M. Surface plasmon resonance imaging for affinity-based biosensors. Biosens Bioelectron. 2010, 25: 957-66), and can be calculated using, e.g., a dissociation constant, K_{D}, such that a lower K_{D} reflects a higher affinity.

As used herein the term “soluble” refers to the ability of the molecules of the present invention to bind an Arenavirus (according to the above measures) under physiological conditions.

As used herein the term “isolated polypeptide” refers to at least partially separated from the natural environment e.g., the human body. According to one embodiment, the isolated polypeptide is essentially free from contaminating cellular components, such as carbohydrates, lipids, or other proteinaceous impurities associated with the polypeptide in nature. Typically, a preparation of the isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The term “polypeptide” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which may have, for example, modifications rendering the polypeptides more stable while in a body or more capable of penetrating into cells.

Such modifications include, but are not limited to N terminus modification, C terminus modification, polypeptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

The term “analog” refers to deletion, addition or substitution of one or more amino acid residues. When preparing analogs obtained by substitution of amino acid residues, it is important that the substitutions be selected from those that cumulatively do not substantially change the volume, hydrophobic-hydrophilic pattern and charge of the corresponding portion of the unsubstituted parent polypeptide. Thus, a hydrophobic residue may be substituted with a hydrophilic residue, or vice-versa, as long as the total effect does not substantially change the volume, hydrophobic-hydrophilic pattern and charge of the corresponding unsubstituted parent polypeptide, i.e. as long as the capability of binding an Arenavirus is kept.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (˜CH2-NH—), sulfide bonds (˜CH2-S—), ethylene bonds (˜CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| Ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| Penicillamine | Pen | N-methylpenicillamine | Nmpen |
| γ-aminobutyric acid | Gabu | N-methyl-γ-aminobutyrate | Nmgabu |
| Cyclohexylalanine | Chexa | N-methyl-cyclohexylalanine | Nmchexa |
| Cyclopentylalanine | Cpen | N-methyl-cyclopentylalanine | Nmcpen |
| α-amino-α-methylbutyrate | Aabu | N-methyl-α-amino-α-methylbutyra [symbol] | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2- | Nmbc | 1,2,3,4-tetrahydroisoquinoline-3- | Tic |
| diphenylethylamino)cyclopropane | | carboxylic acid | |
| Phosphoserine | pSer | phosphothreonine | pThr |
| Phosphotyrosine | pTyr | O-methyl-tyrosine | |
| 2-aminoadipic acid | | hydroxylysine | |

[symbol]

indicates data missing or illegible when filed

According to one embodiment, the amino acid is an unnatural amino acid (also referred to as non-standard amino acid). Examples of unnatural amino acids, without limiting to, are D-amino acids, alpha, alpha-disubstituted amino acids, N-alkyl amino acids, lactic acid, 4-hydroxyproline, y-carboxyglutamate, epsilon-N,N,N-tri methyllysine, epsilon-N-acetyllysine, 0-phosphoserine, N-acetylserine, N-formylmethionine, 3-methylhistidine, 5-hydroxylysine, omega-N-methylarginine, and isoaspartic acid.

According to one embodiment, the amino acid is an “equivalent amino acid residue”. An equivalent amino acid residue refers to an amino acid residue capable of replacing another amino acid residue in a polypeptide without substantially altering the structure and/or functionality of the polypeptide (e.g. capability of binding an Arenavirus). Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

Within the meaning of the term “equivalent amino acid substitution” one amino acid may be substituted for another within the groups of amino acids indicated herein below:
i) Amino acids having polar side chains (Asp, Glu, Lys, Arg, His, Asn, Gln, Ser, Thr, Tyr, Cys);
ii) Amino acids having non-polar side chains (Gly, Ala, Val, Leu, Ile, Phe, Trp, Pro, Met);
iii) Amino acids having non-polar aliphatic side chains (Gly, Ala, Val, Leu, Ile);
iv) Amino acids having cyclic side chains (Phe, Tyr, Trp, His, Pro);
v) Amino acids having aromatic side chains (Phe, Tyr, Trp);
vi) Amino acids having acidic side chains (Asp, Glu);
vii) Amino acids having basic side chains (Lys, Arg, His);
viii) Amino acids having amide side chains (Asn, Gln);
ix) Amino acids having hydroxy side chains (Ser, Thr);
x) Amino acids having sulphur-containing side chains (Cys, Met);
xi) Neutral, weakly hydrophobic amino acids (Pro, Ala, Gly, Ser, Thr);
xii) Hydrophilic amino acids (Arg, Asn, Asp, Glu, Gln, His, Lys, Ser, Thr, Tyr); and
xiii) Hydrophobic amino acids (Ala, Cys, Gly, Ile, Leu, Met, Phe, Pro, Trp, Val).
xiv) Charged amino acids (Arg, Lys, Asp, Glu)

According to a specific embodiment, the polypeptide comprises the amino acid sequence of TfR1 as set forth in SEQ ID NO: 2, 4, 10, 12, 14 or 21.

According to a specific embodiment, the polypeptide comprises the amino acid sequence of human TfR1 apical domain as set forth in SEQ ID NO: 16.

According to a specific embodiment, the polypeptide comprises the amino acid sequence of White-throated woodrat TfR1 apical domain as set forth in SEQ ID NO: 18.

According to one embodiment, the polypeptide is an “active variant” or “functional homolog” which refers to any polypeptide derived from a TfR1 polypeptide sequence, e.g. as set forth in SEQ ID NOs: 2, 4, 10, 12 and 14, and which comprises at least one amino-acid substitution, and which retains at least about 70%, 80%, 90%, 95%, or 100% of the biological activity (e.g. capability of binding an Arenavirus) of the sequence from which it was derived, or to which it is most similar to. These terms also encompass polypeptides comprising regions having substantial similarity to the polypeptide such as structural variants.

The term “substantial similarity” means that two polypeptide sequences, when optimally aligned, share at least about 50%, 60%, 70%, 80%, 90%, 95%, 99% or 100% sequence identity.

According to one embodiment, the polypeptide of some embodiments of the invention comprises a modification (e.g. deletion, insertion or point mutation) in at least one amino acid.

According to one embodiment, the polypeptide comprises a modification (e.g. deletion, insertion or point mutation) in one, two, three, four, five, six, seven, eight, nine, ten or more amino acids, as long as the activity of the polypeptide is retained (e.g. capability of binding an Arenavirus).

Since the present polypeptides are preferably utilized in therapeutics or diagnostics which require the polypeptides to be in soluble form, the polypeptides of some embodiments of the invention may include one or more non-natural or natural polar amino acids, including but not limited to serine and threonine which are capable of increasing polypeptide solubility due to their hydroxyl-containing side chain. Furthermore, the amino acid sequence of the polypeptides of some embodiments of the invention may comprise at least one deletion, insertion or point mutation that renders the TfR1 soluble.

According to one embodiment, the amino acid sequence of the isolated polypeptide comprises a point mutation at an interface between the apical domain and the protease-like domain of the TfR1.

According to one embodiment, the amino acid sequence of the isolated polypeptide comprises a point mutation which abolishes a glycosylation site of the TfR1.

According to one embodiment, the glycosylation site comprises an N—X—S glycosylation motif.

According to one embodiment, the isolated polypeptide comprises a modification in a N—X—S glycosylation motif.

According to one embodiment, Serine of the N—X—S glycosylation motif is mutated to any amino acid or mimetic thereof with the proviso that the amino acid is not Threonine.

According to one embodiment, Serine of the N—X—S glycosylation motif is mutated to Alanine or mimetic thereof.

According to one embodiment, the modification is at Serine 206.

According to a specific embodiment, Serine 206 is modified to Alanine or mimetic thereof. An exemplary amino acid sequence of the isolated polypeptide is set forth in SEQ ID NO: 23

According to one embodiment, Asparagine of the N—X—S glycosylation motif is mutated to any amino acid or mimetic thereof with the proviso that the amino acid not Asparagine.

According to one embodiment, the modification is at Asparagine 204.

According to one embodiment, the amino acid sequence of the isolated polypeptide comprises a substitution of at least one hydrophobic residue with a hydrophilic residue (exemplary hydrophobic and hydrophilic residues which can be substituted according to the present teachings are described hereinabove).

According to one embodiment, the amino acid sequence of the isolated polypeptide comprises a substitution of at least one hydrophobic residue with a charged residue (exemplary hydrophobic and charged residues which can be substituted according to the present teachings are described hereinabove).

According to one embodiment, the amino acid sequence of the isolated polypeptide comprises a substitution of at least one large hydrophobic residue (i.e. Leu or Phe) with a small hydrophobic residue (i.e. Ala or Gly).

According to one embodiment, the amino acid sequence of the isolated polypeptide comprises a substitution of two, three, four, five, six, seven, eight, nine, ten or more hydrophobic residues with hydrophilic residues (exemplary hydrophobic and hydrophilic residues which can be substituted according to the present teachings are described hereinabove).

According to a specific embodiment, the amino acid sequence of the isolated polypeptide comprises a substitution of five hydrophobic residues with hydrophilic residues (exemplary hydrophobic and hydrophilic residues which can be substituted according to the present teachings are described hereinabove).

According to a specific embodiment, the amino acid sequence of the isolated polypeptide comprises a substitution of a hydrophobic residue with a hydrophilic residue corresponding to at any of residues corresponding to residues 283, 288, 291, 295 and/or 298 of SEQ ID NO: 2.

According to a specific embodiment, the amino acid sequence of the isolated polypeptide comprises a substitution of a Methionine with a Lysine at a residue corresponding to residue 283 of SEQ ID NO: 2.

According to a specific embodiment, the amino acid sequence of the isolated polypeptide comprises a substitution of a Phenylalanine with a Tyrosine at a residue corresponding to residue 288 of SEQ ID NO: 2.

According to a specific embodiment, the amino acid sequence of the isolated polypeptide comprises a substitution of a Valine with a Serine at a residue corresponding to residue 291 of SEQ ID NO: 2.

According to a specific embodiment, the amino acid sequence of the isolated polypeptide comprises a substitution of an Isoleucine with a Glutamic acid at a residue corresponding to residue 295 of SEQ ID NO: 2.

According to a specific embodiment, the amino acid sequence of the isolated polypeptide comprises a substitution of a Phenylalanine with a Glutamine at a residue corresponding to residue 298 of SEQ ID NO: 2.

According to a specific embodiment, the amino acid sequence of the isolated polypeptide comprises all the above described substitution of hydrophobic residues with hydrophilic residues.

According to one embodiment, the amino acid sequence of the isolated polypeptide is devoid of the long loop of TfR1.

According to a specific embodiment, the amino acid sequence of the isolated polypeptide is devoid of residues corresponding to residues 301-326 (i.e. long loop) of SEQ ID NO: 2.

It will be appreciated that a short segment in the TfR1 apical domain (i.e. corresponding to residues 208-212 of SEQ ID NO: 2) is a critical determinant of virus host specificity. Accordingly, the polypeptide of some embodiments of the invention does not comprise a modification at amino acid residues corresponding to residues 208-212 of SEQ ID NO: 2.

According to one embodiment, the polypeptide does not comprise a modification at a residue corresponding to Tyrosine 211 (or in any residue flanking this residue) of SEQ ID NO: 2.

According to one embodiment, the isolated polypeptide of some embodiments of the invention comprises up to 50 amino acids, up to 75 amino acids, up to 100 amino acids, up to 110 amino acids, up to 120 amino acids, up to 130 amino acids, up to 140 amino acids, up to 150 amino acids, up to 160 amino acids, up to 170 amino acids, up to 175 amino acids, up to 180 amino acids, up to 185 amino acids, up to 190 amino acids, up to 195 amino acids, up to 200 amino acids, up to 210 amino acids, up to 220 amino acids, up to 230 amino acids, up to 240 amino acids, up to 250 amino acids, up to 260 amino acids, up to 270 amino acids, up to 280 amino acids, up to 290 amino acids, up to 300 amino acids, up to 350 amino acids, or up to 400 amino acids.

According to some embodiments of the invention, the isolated polypeptide is of a length not exceeding 170 amino acids residues.

According to some embodiments of the invention, the isolated polypeptide is of a length not exceeding 175 amino acids residues.

According to some embodiments of the invention, the isolated polypeptide is of a length not exceeding 180 amino acids residues.

According to some embodiments of the invention, the isolated polypeptide is of a length not exceeding 185 amino acids residues.

According to some embodiments of the invention, the isolated polypeptide is of a length not exceeding 195 amino acids residues.

According to one embodiment, the isolated polypeptide of some embodiments of the invention comprises 50-75 amino acids, 50-100 amino acids, 50-150 amino acids, 50-200 amino acids, 50-300 amino acids, 50-400 amino acids, 75-100 amino acids, 75-125 amino acids, 75-150 amino acids, 75-175 amino acids, 75-200 amino acids, 100-125 amino acids, 100-150 amino acids, 100-175 amino acids, 100-200 amino acids, 100-300 amino acids, 100-400 amino acids, 125-150 amino acids, 125-175 amino acids, 125-200 amino acids, 125-250 amino acids, 150-175 amino acids, 150-200 amino acids, 150-250 amino acids, 150-300 amino acids, 150-400 amino acids, 200-250 amino acids, 200-300 amino acids, 200-400 amino acids, 250-300 amino acids, 300-400 amino acids, or 350-400 amino acids.

According to some embodiments of the invention, the isolated polypeptide is 160-180 amino acids in length.

According to some embodiments of the invention, the isolated polypeptide is 160-190 amino acids in length.

According to some embodiments of the invention, the isolated polypeptide is 170-180 amino acids in length.

According to some embodiments of the invention, the isolated polypeptide is 170-175 amino acids in length.

The isolated polypeptides of some embodiments of the invention may be utilized in a linear form, although it will be appreciated that in cases where cyclization does not severely interfere with polypeptide characteristics, cyclic forms of the polypeptide can also be utilized.

According to another embodiment, the polypeptide comprises a protecting moiety or a stabilizing moiety.

The term “protecting moiety” refers to any moiety (e.g. chemical moiety) capable of protecting the polypeptide from adverse effects such as proteolysis, degradation or clearance, or alleviating such adverse effects.

The term “stabilizing moiety” refers to any moiety (e.g. chemical moiety) that inhibits or prevents a polypeptide from degradation.

The addition of a protecting moiety or a stabilizing moiety to the polypeptide typically results in masking the charge of the polypeptide terminus, and/or altering chemical features thereof, such as, hydrophobicity, hydrophilicty, reactivity, solubility and the like. Examples of suitable protecting moieties can be found, for example, in Green et al., “Protective Groups in Organic Chemistry”, (Wiley, 2.sup.nd ed. 1991) and Harrison et al., “Compendium of Synthetic Organic Methods”, Vols. 1-8 (John Wiley and Sons, 1971-1996).

The protecting moiety (or group) or stabilizing moiety (or group) may be added to the N-(amine) terminus and/or the C-(carboxyl) terminus of the polypeptide.

Representative examples of N-terminus protecting/stabilizing moieties include, but are not limited to, formyl, acetyl (also denoted herein as “Ac”), trifluoroacetyl, benzyl, benzyloxycarbonyl (also denoted herein as “CBZ”), tert-butoxycarbonyl (also denoted herein as “BOC”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “FMOC”), nitro-veratryloxycarbonyl (also denoted herein as “NVOC”), t-amyloxycarbonyl, adamantyloxycarbonyl, and p-methoxybenzyloxycarbonyl, 2-chlorobenzyloxycarbonyl and the like, nitro, tosyl (CH3C6H4SO2-), adamantyloxycarbonyl, 2,2,5,7,8-pentamethylchroman-6-sulfonyl, 2,3,6-trimethyl-4-methoxyphenylsulfonyl, t-butyl benzyl (also denoted herein as “BZL”) or substituted BZL, such as, p-methoxybenzyl, p-nitrobenzyl, p-chlorobenzyl, o-chlorobenzyl, 2,6-dichlorobenzyl, t-butyl, cyclohexyl, cyclopentyl, benzyloxymethyl (also denoted herein as “BOM”), tetrahydropyranyl, chlorobenzyl, 4-bromobenzyl, and 2,6-dichlorobenzyl.

According to one embodiment of the invention, the protecting/stabilizing moiety is an amine protecting moiety.

According to a specific embodiment, the protecting/stabilizing moiety is a terminal cysteine residue.

Representative examples of C-terminus protecting/stabilizing moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus may be modified to an amide group.

Other modifications of polypeptides include replacement of the amine and/or carboxyl with a different moiety, such as hydroxyl, thiol, halide, alkyl, aryl, alkoxy, aryloxy and the like.

According to a specific embodiment, the protecting/stabilizing moiety is an amide.

According to a specific embodiment, the protecting/stabilizing moiety is a terminal cysteine residue.

According to one embodiment, the protecting/stabilizing moiety comprises at least one, two, three or more cysteine residues at the N- or C-termini of the polypeptide.

According to one embodiment, the protecting/stabilizing moiety comprises one cysteine residues at the N- or C-termini of the polypeptide.

According to one embodiment, the protecting/stabilizing moiety comprises at least one, two, three or more cysteine residues at both the N- and C-termini of the polypeptide.

According to one embodiment, the protecting/stabilizing moiety comprises one cysteine residue at both the N- and C-termini of the polypeptide.

Additionally or alternatively, the polypeptide of the invention may further comprise a protease-disabling moiety. Such a moiety is capable of binding to a protease and transiently or permanently disabling its proteolytic activity.

In some embodiments, the protease-disabling moiety may be an irreversible inhibitor selected from the group consisting of substituted acetyl (1-x-actyl), sulfonylfluorides (—SO2F), chloromethylketones (—COCH2CI), esters (—COOR), boronic acids (—B(OR)2) and combinations thereof.

In some embodiments, the protease-disabling moiety may be a reversible inhibitor selected from the group consisting of aldehydes (—CHO), arylketones (—CO-Aryl), trifluoromethylketones (—COCF3) ketocarboxylic acids (—COCOOH) and combinations thereof.

In some embodiments the protease-disabling moiety may be a protease-disabling compound selected from the group consisting of chloromethyiketone (CK) and derivatives thereof, sulfonylfluorides (—SO2F), chloromethylketones (—COCH2CII), esters (—COOR), boronic acids (—B(OR)2), aldehydes (—CHO), arylketones (—CO-Aryl), trifluoromethylketones (—COCF3) and ketocarboxylic acids (—COCOOH).

In some embodiments, the protease-disabling moiety may be a substituted acetyl. In some embodiments, the substituted acetyl may be haloacetyl. In some embodiments, the haloacetyl may be chloroacetyl. In some embodiments, the protease-disabling moiety may be chloromethylketone (CK).

In one embodiment, the polypeptides are modified only at the N-termini or the C-termini thereof (e.g. resulting in a molecule that has a negative net charge or a positive net charge, respectively). In another embodiment, the polypeptides are modified at both the N-termini and the C-termini (e.g. resulting in uncharged molecules).

According to one embodiment, the moiety is bound to the amino acid sequence of the polypeptide directly or via a linker.

According to a specific embodiment, the isolated soluble polypeptide comprising the protecting moiety and/or a stabilizing moiety is the polypeptide comprising the amino acid sequence set forth in SEQ ID NO: 6.

According to one embodiment, there is provided a composition of matter comprising a soluble polypeptide comprising an amino acid sequence of a TfR1 apical domain (also termed sAD) as set forth in SEQ ID NO: 6, the soluble polypeptide being capable of binding an Arenavirus.

Also included in the scope of the present invention are “chemical derivative” of a polypeptide or analog. Such chemical derivates contain additional chemical moieties not normally a part of the polypeptide. Covalent modifications of the polypeptide are included within the scope of this invention. Such modifications may be introduced into the molecule by reacting targeted amino acid residues of the polypeptide with an organic derivatizing agent that is capable of reacting with selected side chains or terminal residues. Many such chemical derivatives and methods for making them are well known in the art, some are discussed hereinbelow.

Also included in the scope of the invention are salts of the polypeptides and analogs of the invention. As used herein, the term “salts” refers to both salts of carboxyl groups and to acid addition salts of amino groups of the polypeptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids, such as, for example, acetic acid or oxalic acid. Such chemical derivatives and salts are preferably used to modify the pharmaceutical properties of the polypeptide insofar as stability, solubility, etc., are concerned.

According to one embodiment of the invention, the isolated polypeptide capable of binding an Arenavirus (i.e., the polypeptide described herein) is attached to a heterologous moiety.

As used herein the phrase “heterologous moiety” refers to an amino acid sequence which does not endogenously form a part of the isolated polypeptide's amino acid sequence. Preferably, the heterologous moiety does not affect the biological activity of the isolated polypeptide (e.g. capability of binding an Arenavirus).

The heterologous moiety may thus serve to ensure stability of the isolated polypeptide of the present invention without compromising its activity. For example, the heterologous polypeptide may increase the half-life of the isolated polypeptide or molecule in the serum.

The heterologous moiety of the present invention may be capable of inducing an antibody dependent cellular-mediated cytotoxicity (ADCC) response as discussed in detail hereinbelow.

According to one embodiment, the heterologous moiety does not induce an immune response. Thus, for instance, in the case of Ig, it may contain human sequences that do not produce an immune response in a subject administered therewith.

According to one embodiment, the heterologous moiety is for increasing avidity of the polypeptide.

According to one embodiment, the heterologous moiety is for multimerization of the isolated polypeptide (e.g. at least for dimerization of the isolated polypeptides).

According to one embodiment, the heterologous moiety is a proteinaceous moiety.

Examples of heterologous amino acid sequences that may be used in accordance with the teachings of the present invention include, but are not limited to, immunoglobulin, galactosidase, glucuronidase, glutathione-S-transferase (GST), carboxy terminal peptide (CTP) from chorionic gonadotrophin (CGb) and chloramphenicol acetyltransferase (CAT) [see for example U.S. Publication No. 20030171551].

According to a specific embodiment, the heterologous amino acid sequence is an immunoglobulin.

Generally the heterologous amino acid sequence is localized at the amino- or carboxyl-terminus (N-ter or C-ter, respectively) of the isolated polypeptide of the present invention. The heterologous amino acid sequence may be attached to the isolated polypeptide amino acid sequence by any of peptide or non-peptide bond. Attachment of the isolated polypeptide amino acid sequence to the heterologous amino acid sequence may be effected by direct covalent bonding (peptide bond or a substituted peptide bond) or indirect binding such as by the use of a linker having functional groups. Functional groups include, without limitation, a free carboxylic acid (C(═O)OH), a free amino group (NH₂), an ester group (C(═O)OR, where R is alkyl, cycloalkyl or aryl), an acyl halide group (C(═O)A, where A is fluoride, chloride, bromide or iodide), a halide (fluoride, chloride, bromide or iodide), a hydroxyl group (OH), a thiol group (SH), a nitrile group (C≡N), a free C-carbamic group (NR″—C(═O)—OR′, where each of R′ and R″ is independently hydrogen, alkyl, cycloalkyl or aryl).

An example of a heterologous amino acid sequence which may be used in accordance with this aspect of the present invention is an immunoglobulin amino acid sequence, such as the hinge and Fc regions of an immunoglobulin heavy domain (see U.S. Pat. No. 6,777,196). The immunoglobulin moiety in the molecules of this aspect of the present invention may be obtained from IgG1, IgG2, IgG3 or IgG4 subtypes, IgA, IgE, IgD or IgM, as further discussed hereinbelow.

Typically, in such fusions the chimeric molecule will retain at least functionally active hinge and CH2 and CH3 domains of the constant region of an immunoglobulin heavy chain. Fusions can also be generated to the C-terminus of the Fc portion of a constant domain, or immediately N-terminal to the CH1 of the heavy chain or the corresponding region of the light chain.

Though it may be possible to conjugate the entire heavy chain constant region to the isolated polypeptide amino acid sequence of the present invention, it is preferable to fuse shorter sequences. For example, a sequence beginning at the hinge region upstream of the papain cleavage site, which defines IgG Fc chemically; residue 216, taking the first residue of heavy chain constant region to be 114, or analogous sites of other immunoglobulins, may be used in the fusion. In a particular embodiment, the isolated polypeptide's amino acid sequence is fused to the hinge region and CH2 and CH3, or to the CH1, hinge, CH2 and CH3 domains of an IgG1, IgG2, or IgG3 heavy chain (see U.S. Pat. No. 6,777,196).

As mentioned, the immunoglobulin sequences used in the construction of the chimeric molecules of this aspect of the present invention may be from an IgG immunoglobulin heavy chain constant domain. Such IgG immunoglobulin sequence can be purified efficiently on, for example, immobilized protein A. Selection of a fusion partner may also take into account structural and functional properties of immunoglobulins. Thus, for example, the heterologous peptide may be IgG3 hinge which is longer and more flexible, so it can accommodate larger amino acid sequences that may not fold or function properly when fused to IgG1. Another consideration may be valency; IgG are bivalent homodimers, whereas Ig subtypes like IgA and IgM may give rise to dimeric or pentameric structures, respectively, of the basic Ig homodimer unit. Other considerations in selecting the immunoglobulin portion of the chimeric molecules of this aspect of the present invention are described in U.S. Pat. No. 6,777,196.

The molecules of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463.

The heterologous moiety may also be chemically linked to the isolated polypeptide following the independent generation of each. Thus, the two polypeptides may be covalently or non-covalently linked using any linking or binding method and/or any suitable chemical linker known in the art. Such linkage can be direct or indirect, as by means of a peptide bond or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched, or cyclic side chains, internal carbon or nitrogen atoms, and the like. The exact type and chemical nature of such cross-linkers and cross linking methods is preferably adapted to the type and nature of the peptides used.

According to one embodiment, there is provided a fusion protein comprising an amino acid sequence of a TfR1 apical domain and an amino acid sequence of IgG Fc, the fusion protein capable of binding an Arenavirus.

As used herein, the term “fused” means that at least a protein or peptide is physically associated with another protein or peptide, which naturally don't form a complex. According to a specific embodiment the fused molecule is a “fusion polypeptide” or “fusion protein”, a protein created by joining two or more heterologously related polypeptide sequences together. The fusion polypeptides encompassed in this invention include translation products of a chimeric nucleic acid construct that joins the DNA sequence encoding a TfR1 apical domain with the DNA sequence encoding an IgG Fc to form a single open-reading frame. In other words, a “fusion polypeptide” or “fusion protein” is a recombinant protein of two or more proteins which are joined by a peptide bond.

The terms “fusion protein”, “chimera”, “chimeric molecule”, or “chimeric protein” are used interchangeably.

According to a specific embodiment, the fusion protein (termed Arenacept) is as set forth in SEQ ID NO: 8.

According to a specific embodiment, the fusion protein (termed Arenacept^{S206A}) is as set forth in SEQ ID NO: 23.

Thus, the molecule of this aspect of the present invention may comprise a heterologous moiety, as described above. Additionally or alternatively, the isolated polypeptide's amino acid sequence of the present invention may be attached to a non-proteinaceous moiety.

The phrase “non-proteinaceous moiety” as used herein refers to a molecule, not including peptide bonded amino acids, that is attached to the above-described isolated polypeptide's amino acid sequence.

According to one embodiment, the non-proteinaceous moiety is non-toxic.

Exemplary non-proteinaceous moieties which may be used according to the present teachings include, but are not limited to, polyethylene glycol (PEG), Polyvinyl pyrrolidone (PVP), poly(styrene comaleic anhydride) (SMA), and divinyl ether and maleic anhydride copolymer (DIVEMA).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant polypeptide product is subjected to in-vitro modification (e.g., PEGylation as further described hereinbelow).

It will be appreciated that such non-proteinaceous moieties may also be attached to the above mentioned fusion molecules (i.e., which comprise a TfR1 apical domain and an amino acid sequence of IgG Fc, the fusion molecules capable of binding an Arenavirus) to promote stability and possibly solubility of the molecules.

Bioconjugation of such a non-proteinaceous moiety (such as PEGylation) can confer the isolated polypeptide's or fusion protein's amino acid sequence with stability (e.g., against protease activities) and/or solubility (e.g., within a biological fluid such as blood, digestive fluid) while preserving its biological activity and prolonging its half-life.

Bioconjugation is advantageous particularly in cases of therapeutic proteins which exhibit short half-life and rapid clearance from the blood. The increased half-lives of bioconjugated proteins in the plasma results from increased size of protein conjugates (which limits their glomerular filtration) and decreased proteolysis due to polymer steric hindrance. Generally, the more polymer chains attached per polypeptide, the greater the extension of half-life. However, measures are taken not to reduce the specific activity of the isolated polypeptide or fusion protein of the present invention (e.g. capability of binding an Arenavirus).

Bioconjugation of the isolated polypeptide's or fusion protein's amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form.

In general, the PEG added to the isolated polypeptide's or fusion protein's amino acid sequence of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the isolated polypeptide's or fusion protein's amino acid sequence by site-specific mutagenesis as long as the activity of the conjugate is retained (e.g. capability of binding an Arenavirus). A target for PEGylation could be any Cysteine residue at the N-terminus or the C-terminus of the isolated polypeptide's or fusion protein's amino acid sequence. Additionally or alternatively, other Cysteine residues can be added to the isolated polypeptide's or fusion protein's amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1:acryloyl chloride 1.5:triethylamine 2, at 0.2 gram PEG/mL DCM). Such chemical groups can be attached to linearized, 2-arm, 4-arm, or 8-arm PEG molecules.

While conjugation to cysteine residues is one convenient method by which the isolated polypeptide's or fusion protein's amino acid of the present invention can be PEGylated, other residues can also be used if desired. For example, acetic anhydride can be used to react with NH₂ and SH groups, but not COOH, S—S, or —SCH₃ groups, while hydrogen peroxide can be used to react with —SH and —SCH₃ groups, but not NH₂. Reactions can be conducted under conditions appropriate for conjugation to a desired residue in the polypeptide employing chemistries exploiting well-established reactivities.

For bioconjugation of the isolated polypeptide's or fusion protein's amino acid sequence of the present invention with PVP, the terminal COOH-bearing PVP is synthesized from N-vinyl-2-pyrrolidone by radical polymerization in dimethyl formamide with the aid of 4,4′-azobis-(4-cyanovaleric acid) as a radical initiator, and 3-mercaptopropionic acid as a chain transfer agent. Resultant PVPs with an average molecular weight of Mr 6,000 can be separated and purified by high-performance liquid chromatography and the terminal COOH group of synthetic PVP is activated by the N-hydroxysuccinimide/dicyclohexyl carbodiimide method. The isolated polypeptide's or fusion protein's amino acid sequence is reacted with a 60-fold molar excess of activated PVP and the reaction is stopped with amino caploic acid (5-fold molar excess against activated PVP), essentially as described in Haruhiko Kamada, et al., 2000, Cancer Research 60: 6416-6420, which is fully incorporated herein by reference.

Resultant conjugated isolated polypeptide or fusion protein molecules (e.g., PEGylated or PVP-conjugated isolated polypeptide or fusion protein) are separated, purified and qualified using e.g., high-performance liquid chromatography (HPLC). In addition, purified conjugated molecules of this aspect of the present invention may be further qualified using e.g., in vitro assays in which the binding specificity of isolated polypeptide or fusion protein to its ligand (e.g., Arenavirus) is tested in the presence or absence of the isolated polypeptide or fusion protein conjugates of the present invention, essentially as described for other polypeptides e.g. by surface plasmon resonance assay.

Molecules of this aspect of present invention can be biochemically synthesized such as by using standard solid phase techniques. These methods include exclusive solid phase synthesis, partial solid phase synthesis methods, fragment condensation and classical solution synthesis. These methods are preferably used when the polypeptide is relatively short (i.e., 10 kDa) and/or when it cannot be produced by recombinant techniques (i.e., not encoded by a nucleic acid sequence) and therefore involve different chemistry.

Thus, the polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage.

The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the polypeptide compounds of some embodiments of the invention involves solid phase peptide synthesis.

Large scale peptide synthesis is described by Andersson Biopolymers 2000; 55(3):227-50.

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the polypeptides of the present invention are desired, the polypeptides of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463.

For example, a nucleic acid sequence encoding an isolated polypeptide of the present invention (e.g., the amino acid sequences set forth in SEQ ID NOs: 2, 4, 6, 10, 12 or 14) is ligated to a nucleic acid sequence which may include an inframe sequence encoding a proteinaceous moiety such as immunoglobulin.

According to one embodiment, the nucleic acid sequence encodes a fusion protein (e.g. Arenacept, as set forth in SEQ ID NO: 8 or as set forth in SEQ ID NO: 23).

For expression, a nucleic acid sequence encoding an isolated polypeptide may comprise the nucleic acid sequence as set forth in SEQ ID NO: 1, 3, 5, 9, 11, 13 or 20.

According to one embodiment, a nucleic acid sequence encoding a fusion protein (e.g. Arenacept) may comprise the nucleic acid sequence as set forth in SEQ ID NO: 7 or as set forth in SEQ ID NO: 22.

Also provided is an expression vector, comprising the isolated polynucleotide of some embodiments of the invention. According to one embodiment, the polynucleotide sequence is operably linked to a cis-acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion or presentation of antibody from a host cell in which it is placed. Preferably the signal sequence for this purpose is a mammalian signal sequence.

According to one embodiment, the signal peptide is as set forth in SEQ ID NO: 19.

Preferably, the promoter utilized by the nucleic acid construct of some embodiments of the invention is active in the specific cell population transformed.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of TCRL mRNA translation.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Improvements in recombinant polypeptide expression in mammalian cells can be achieved by effectively increasing the gene dosage in a transfected host cell. Increases in gene copy number are most commonly achieved by gene amplification using cell lines deficient in an enzyme such as dihydrofolate reductase (DHFR) or glutamine synthetase (GS) in conjunction with expression vectors containing genes encoding these enzymes and agents such as methotrexate (MTX), which inhibits DHFR, and methionine sulfoxamine (MSX), which inhibits GS.

Exemplary systems for expression are described in EP2861741, US20120178126, and US20080145895, each of which is incorporated herein by reference in its entirety.

Also provided are cells which comprise the polynucleotides/expression vectors as described herein.

Suitable host cells for cloning or expression include prokaryotic or eukaryotic cells. See e.g. Charlton, Methods in Molecular Biology, Vol. 248 (B. K. C. Lo, ed., Humana Press, Totowa, N.J., 2003), pp. 245-254, describing expression of antibody fragments in E. coli; see Gerngross, Nat. Biotech. 22:1409-1414 (2004), and Li et al., Nat. Biotech. 24:210-215 (2006) for suitable fungi and yeast strains; and see e.g., U.S. Pat. Nos. 5,959,177, 6,040,498, 6,420,548, 7,125,978, and 6,417,429 for suitable plant cell cultures which can also be utilized as hosts.

After expression, the isolated polypeptide or fusion protein may be isolated from the cells in a soluble fraction and can be further purified.

Recovery of the isolated polypeptide or fusion protein may be effected following an appropriate time in culture. The phrase “recovering the recombinant polypeptide or fusion protein” refers to collecting the whole fermentation medium containing the polypeptide or fusion protein and need not imply additional steps of separation or purification.

Notwithstanding the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Molecules of the present invention are preferably retrieved in “substantially pure” form. As used herein, “substantially pure” refers to a purity that allows for the effective use of the protein in the applications, described herein.

It will be appreciated that the composition of matter comprising the isolated polypeptide or fusion protein of the present invention may comprise a single isolated polypeptide or fusion protein or alternatively may comprise two or more isolated polypeptides or fusion proteins fused together according to any of the methods described hereinabove.

Once polypeptides are obtained, they may be tested for Arenavirus binding affinity as discussed in detail above.

According to one embodiment, the composition of matter comprising the isolated polypeptides or fusion proteins of some embodiments of the invention is also selected capable of neutralizing the Arenaviruses for maximizing therapeutic efficacy.

The term “neutralizing” refers the ability of the composition of matter comprising the isolated polypeptides or fusion proteins to block the site(s) on viruses that they use to enter their target cell. According to one embodiment, the composition of matter comprising the isolated polypeptides or fusion proteins of some embodiments of the invention are capable of neutralizing the virus infectivity by at least 10%, 20%, 30%, 40%, 50%, 60%, 70%, 80%, 90%, or by 100% as compared to infectivity in the absence of the composition of matter comprising the isolated polypeptides or fusion proteins of the invention.

Determination of neutralizing of Arenaviruses can be carried out using any method known in the art, such as, by in vitro neutralization assays (such as the one described in the ‘general materials and experimental procedures section’ below).

According to one embodiment, the composition of matter comprising the isolated polypeptides or fusion proteins of some embodiments of the invention is also selected capable of initiating antibody-dependent cellular cytotoxicity (ADCC), i.e. the killing of an antibody-coated target cell by a cytotoxic effector cell (e.g. NK cells, monocytes, macrophages, neutrophils eosinophils and dendritic cells) through a non-phagocytic process (e.g. by the release of the content of cytotoxic granules or by the expression of cell death-inducing molecules).

Determination that the isolated peptides or fusion proteins initiate ADCC can be carried out using any method known in the art such as by measuring lactate dehydrogenase (LDH) release using LDH Cytotoxicity Detection kit (available e.g. from Roche Applied Science).

According to one embodiment, the composition of matter comprising the isolated polypeptides or fusion proteins of some embodiments of the invention is typically capable of promoting eradication of infected cells as well as directly neutralizing Arenaviruses.

According to one embodiment, the composition of matter comprising the isolated polypeptides or fusion proteins of some embodiments of the invention is also selected thermo-stable (e.g. stable up to 45° C., up to 50° C., up to 55° C., up to 60° C., or even up to 65° C.). Such determinations can be carried out using any method known in the art, such as by circular dichroism measurements (such as described in the ‘general materials and experimental procedures section’ below).

The composition of matter comprising the isolated polypeptides or fusion proteins of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition of matter comprising the isolated polypeptides or fusion proteins accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (composition of matter comprising the isolated polypeptides or fusion proteins) effective to prevent, alleviate or ameliorate symptoms of a disorder (e.g., Arenaviral infection) or prolong the survival of the subject being treated.

According to an embodiment of the present invention, an effective amount of the composition of matter comprising the isolated polypeptides or fusion proteins of some embodiments of the present invention is an amount selected to neutralize Arenaviruses and/or eliminate infected cells e.g. by initiating ADCC.

Determination of a therapeutically effective amount is well within the capability of those skilled in the art, especially in light of the detailed disclosure provided herein.

For example, any in vivo or in vitro method of evaluating Arenavirus viral load may be employed.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It will be appreciated that the kit may further comprise another therapeutic composition for treating an Arenavirus infection, e.g. antiviral agent. Thus, for example, the composition of matter comprising the isolated polypeptide or fusion protein can be packaged in one container while the antiviral agent may be packaged in a second container both for therapeutic treatment. Alternatively, the composition of matter comprising the isolated polypeptide or fusion protein can be packaged in a co-formulation with the antiviral agent.

As mentioned above, the composition of matter comprising the isolated polypeptides or fusion proteins of the invention specifically target Arenaviruses. Thus, the composition of matter comprising the isolated polypeptides or fusion proteins can be used to treat or prevent an Arenavirus viral infection or disease associated therewith (as discussed below).

According to another aspect of the invention, there is provided a method of treating or preventing an Arenavirus viral infection or disease associated therewith in a subject in need thereof, the method comprising administering to the subject a therapeutically effective amount of the composition of matter comprising an isolated soluble polypeptide comprising an amino acid sequence of a TfR1 apical domain or fusion protein of some embodiments of the invention, thereby treating or preventing the Arenavirus viral infection or disease associated therewith in the subject.

According to another aspect, there is provided a composition of matter comprising an isolated soluble polypeptide comprising an amino acid sequence of a TfR1 apical domain or fusion protein of some embodiments of the invention, for use in treating or preventing an Arenavirus viral infection or disease associated therewith in a subject in need thereof.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings, male or female, at any age or gender, which suffer from the pathology. Preferably, this term encompasses individuals who are at risk to develop the pathology.

As used herein, the phrase “Arenavirus viral infection” refers to any infection caused by an Arenavirus. According to a specific embodiment, the Arenavirus is a New World Arenavirus as described in detail hereinabove.

As used herein, the phrase “disease associated therewith” refers to any disease or symptom caused as a result of the Arenavirus viral infection. These can include, without being limited to, flu-like symptoms (e.g. fever, chills, etc.), vomiting, headaches, muscular rigidity, photophobia, hyperexcitability, abnormal tremors and movements, incoordination, paralysis, sensory loss, convulsions, apathy, memory defects, confusion, mental difficulties, respiratory dysfunction, neuronal damage, vascular damage, bleeding, severe hemorrhages, and hemorrhagic fever.

According to one embodiment, when the disease is a Junin (JUNV) infection, the symptoms may include, for example, conjunctivitis, purpura, petechia and occasionally sepsis.

According to one embodiment, when the disease is a Machupo (MACV) infection, the symptoms may include, for example, fever, headache, fatigue, myalgia, and arthralgia, as well as hemorrhagic signs e.g. bleeding from nasal and oral mucosa, bronchopulmonary, gastrointestinal, and genitourinary tracts.

According to one embodiment, when the disease is a Guanarito (GTOV) infection, the symptoms may include, for example, fever, malaise, headache, arthralgia, sore throat, vomiting, abdominal pain, diarrhea, convulsions, and a variety of hemorrhagic manifestations.

According to one embodiment, when the disease is a Sabia (SABV) infection, the symptoms may include, for example, fever, headache, myalgia, nausea, vomiting, weakness, pronounced sore throat, conjunctivitis, diarrhea, epigastric pain, and bleeding gums.

According to one embodiment, the disease is a hemorrhagic fever.

The isolated soluble polypeptide or fusion protein of the present invention can also be administered with other therapeutically or nutritionally useful agents, such as antibiotics, vitamins, herbal extracts, anti-inflammatories, glucose, antipyretics, analgesics, interleukins (IL-1, IL-2, IL-3, IL-4, IL-5, IL-6, IL-7, IL-8, IL-9, IL-10 IL-11, IL-12, IL-13, IL-14, or IL-15), TPO, or other growth factor such as CSF-1, SF, leukemia inhibitory factor (LIF), or fibroblast growth factor (FGF), as well as C-KIT ligand, M-CSF and TNF-α, PIXY-321 (GM-CSF/IL-3 fusion protein), macrophage inflammatory protein, thrombopoietin, growth related oncogene or chemotherapy and the like. Such determinations are well within the skill of a person of ordinary skill in the art.

As mentioned above, the composition of matter comprising the isolated polypeptide or fusion protein of some embodiments of the invention are suitable for diagnostic applications.

According to an aspect of the present invention, there is provided a method of diagnosing an Arenavirus viral infection in a subject, the method comprising:

(a) contacting a biological sample from the subject with the composition of matter comprising an isolated soluble polypeptide comprising an amino acid sequence of a TfR1 apical domain or fusion protein of some embodiments of the invention, under conditions which allow the formation of immunocomplexes between an Arenavirus and the soluble polypeptide or the fusion protein; and
(b) determining a level of the immunocomplexes in the biological sample, wherein an increase in level of the immunocomplexes beyond a predetermined threshold with respect to a level of the immunocomplexes in a biological sample from a healthy individual is indicative of the Arenavirus viral infection.

As used herein the term “diagnosing” refers to classifying a disease, determining a severity of a disease (grade or stage), monitoring progression, forecasting an outcome of the disease and/or prospects of recovery.

The subject may be a healthy subject (e.g., human) undergoing a routine well-being check-up. Alternatively, the subject may be at risk of the disease or infection. Yet alternatively, the method may be used to monitor treatment efficacy.

As used herein “biological sample” refers to a sample of tissue or fluid isolated from a subject, including but not limited to, for example, plasma, serum, spinal fluid, lymph fluid, the external sections of the skin, respiratory, intestinal, and genitourinary tracts, tears, saliva, sputum, milk, blood cells, tumors, neuronal tissue, organs, and also samples of in vivo cell culture constituents. It should be noted that a “biological sample obtained from the subject” may also optionally comprise a sample that has not been physically removed from the subject, as described in greater detail below.

Numerous well known tissue or fluid collection methods can be utilized to collect the biological sample from the subject in order to determine the level of Arenaviruses or infected cells in the sample. Collections methods include, but are not limited to, fine needle biopsy, needle biopsy, core needle biopsy and surgical biopsy (e.g., brain biopsy), buccal smear and lavage. Regardless of the procedure employed, once a biopsy/sample is obtained the level of the variant can be determined and a diagnosis can thus be made.

As mentioned, the method of the present invention is effected under conditions sufficient to form an immunocomplex (e.g. a complex between the composition of matter comprising the isolated polypeptide or fusion protein of the present invention and the Arenavirus). Such conditions (e.g., appropriate concentrations, buffers, temperatures, reaction times) as well as methods to optimize such conditions are known to those skilled in the art, and examples are disclosed herein below.

The composition of matter comprising the isolated polypeptide or fusion protein of the present invention may comprise e.g., be attached, to an identifiable moiety. Alternatively or additionally, the composition of matter comprising the isolated polypeptide or fusion protein may be identified indirectly such as by using a secondary antibody.

According to one embodiment, diagnosis is corroborated using any diagnostic method known in the art, such as by measuring the viral load or titer, by antigen level measurement, antibody level measurement, virus isolation and/or genomic detection by reverse transcriptase-polymerase chain reaction (RT-PCR), etc. For example, a higher viral load or titre often correlates with the severity of an active viral infection. The quantity of virus per mL can be calculated for example by estimating the live amount of virus in an involved body fluid (e.g. serum sample or whole blood).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NOs: 1, 3, 5 and 7 are expressed in a DNA sequence format (e.g., reciting T for thymine), but they can refer to either a DNA sequence that corresponds to an TfR1 nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Construction of Expression Vectors

Codon optimized forms of MACV, JUNV, GTOV and SBOV glycoprotein complex (GPC) genes have been chemically synthesized (Genescript) according to their UniProt sequences, as follows: MACV (Q6IUF7), JUNV (O10428), GTOV (Q8AYW1) and SBOV (H6V7J2). Genes encoding WWAV and MACV GPC were also provided. All GPCs were subcloned into the pcDNA3.1 expression vector, using BamHI-NotI restriction sites. GP1_{JUNV}Fc, GP1_{MACV}-Fc, GP1GP1_{GTOV}-Fc, GP1_{SBOV}-Fc and GP1_{WWAV}-Fc and sAD-Fc (Arenacept) fusion proteins were generated as previously described [Cohen-Dvashi, et al., J Virol (2015) 89: 7584-7592]. Mutated variant Y211A of Arenacept was generated by PCR using Kapa HiFi DNA polymerase (Kapa Biosystems) according to the QuikChange site-directed mutagenesis manual. Human transferrin receptor encoding vector, hTfR-pENTR221 was obtained from Weizmann Institute Forscheimer plasmid bank, and was subcloned into pQXIP using BamHI-NotI restriction sites.

Protein Expression and Purification

To express and purify the complex of soluble apical domain (sAD) with GP1_{MACV} for structural studies, the present inventors used the same methodologies as used previously for producing GP1_{LASV} [Cohen-Dvashi, et al. (2015) supra]. Briefly, the two proteins were co-expressed as secreted proteins using the baculovirus system in Tni (Trichoplusia ni) cells (Expression Systems). Media were collected and buffer exchanged to TBS (20 mM Tris-HCl pH 8.0, 150 mM sodium chloride) using a tangential flow filtration system (Millipore). Complex was captured using a HiTrap IMAC FF Ni⁺² (GE Healthcare) affinity column followed by size exclusion chromatography purification with a superdex 75 10/300 column (GE Healthcare). Fc-fused GP1s (GP1_{JUNV}-Fc, GP1_{MACV}-Fc, GP1_{GTOV}-Fc, GP1_{SBOV}-Fc and GP1_{WWAV}-Fc) and Arenacept were expressed in HEK293 cells adapted to suspension cells (Expression Systems). Transfections were done using linear 25 kDa polyethylenimine (PEI) (Polysciences) at 1 mg of plasmid DNA per 1 L of culture at cell density of 1 M/ml. Media were collected after 5 days of incubation and supplemented with 0.02% (wt/vol) sodium azide and PMSF. Fusion proteins were isolated using protein-A affinity column (GE Healthcare).

Surface Plasmon Resonance (SPR) Measurements

The binding of NA-sAD to GP1_{JUNV}-Fc, GP1_{MACV}-Fc, GP1_{GTOV}-Fc, GP1_{SBOV}-Fc and GP1_{WWAV}-Fc fusion proteins was measured using a Biacore T200 instrument (GE Healthcare). Fusion proteins were first immobilized at coupling density of approximately 500 resonance units (RU) on a series S sensor chip protein A (GE Healthcare) in TBS and 0.02% sodium azide buffer. One of the four flow cells on the sensor chip was coupled with GP1_{LASV}-Fc to serve as a blank. NA-sAD was then injected at 1000, 500, 250, 50 and 5 nM concentrations, at a flow rate of 80 μL/min. A single cycle kinetics was performed for the binding assay. Sensor chip was regenerated using 10 mM Glycin-HCL pH 1.5 buffer.

In Vitro Neutralization Assays

Pseudoviral particles of MACV, JUNV, GTOV and SBOV were produced as previously described [Cohen-Dvashi et al., J Virol (2016) 90: 10329-10338], except for the use of pLXIN-Luc as the reporter gene (Addgene plasmid #60683). Media containing pseudoviruses were concentrated×10 by PEG precipitation. For that, the viral-containing media were supplemented with PEG 6000 (Sigma) in PBS to a final concentration of 8% (wt/vol). Following incubation of 18 hours at 4° C., viruses were pelleted by centrifugation at 10,000 g for 20 minutes. Pellets of viruses were resuspended in cells media.

For generating a stable cell line that overexpress hTfR, HEK293T cells were transfected with hTfR-pQXIP vector. At 48 hours post transfection, media were replaced to fresh media supplemented with 2 μg/ml puromycin for selection. Cells were grown in the presence of the antibiotics for 1 week. Resistant colonies of stable cells were collected and cultured to form a polyclonal cell line.

For neutralization assays, hTfR-stable HEK293T were seeded on poly-L-Lysine pre-coated white, chimney 96-well plate (Greiner Bio-One). Cells were let to adhere for 2 hours, followed by addition of ×10 concentrated pseudoviruses, which were pre-incubated with 3-fold descending concentrations of either Arenacept or sAD. Cells were washed from viruses at 18 hours post-infection, and luminescence from activity of luciferase was measured at 48 hours post-infection using a TECAN plate reader after applying Bright-Glo reagent (Promega) on cells.

Cell Staining and Fluorescence-Microscopy Imaging

HEK293T cells were seeded on poly-L-Lysine pre-coated cover slips in 24-well plates and transfected with different GPCs using PEI reagent. At 24 hours post transfection, cells were incubated for 5 minutes with 1 μg/ml Arenacept diluted in cell's media, fixed with pre-warmed 3.7% formaldehyde (PFA) solution in PBS and blocked with 3% BSA in PBS. Cells were stained with Cy3-conjugated anti-Human Fc and FITC-conjugated wheat germ agglutinin (WGA). Cells were imaged at ×100 magnification using an Olympus IX83 microscope coupled to a Yokogawa CSU-W1 spinning disc confocal scanner. Images were processed using ImageJ.

Circular Dichroism Measurements

Stock solution of 10 mg/ml sAD in 20 mM Tris-HCl (pH 8.0) 150 mM sodium chloride was diluted 1:40 in 150 mM sodium chloride solution (pH 5.0) for recording circular dichroism (CD) spectra using a Chirascan-plus ACD spectrometer. For determining temperature stability of the protein, CD spectra at wavelength of 222 nm were measured at temperatures ranges between 30 to 85° C. (ramping of 0.5 degree per 5s).

Crystallization

Screening for initial crystallization conditions was done with an 8.8 mg/ml stock of the complex sAD/GP1_{MACV}, using a Mosquito crystallization robot (TTP Labs). Initial hits were identified using the JCSG-plus screen (Molecular Dimensions) and were optimized manually. Crystals were obtained using sitting drop vapor diffusion in 0.2 M Na Thiocyanate pH 6.9, 20% PEG 3350 and 5% MPD. Crystals were then successively cryo-protected using 20% MPD in reservoir solution before flash cooling in liquid nitrogen.

Data Collection, Structure Solution and Refinement

X-ray diffraction data were collected at the European Synchrotron Radiation Facility (ESRF) beamlines ID30B using a Pilatus 6M-F. detector at 100° K. Data to 2.7 Å that appeared to belong to a tetragonal space group was collected. The present inventors used HKL2000 [Otwinowski, et al. Method Enzymol (1997) 276: 307-326] to index, integrate, and scale the data. The present inventors used Phaser [Adams et al., Acta crystallographica. Section D, Biological crystallography (2010) 66: 213-221] to obtain MR solution using the structure of GP1_{MACV} in complex with the apical domain of hTfR1 (PDB: 3KAS), as a search model. Crystal belonged to a tetragonal P 4₃ 2 2 space group, and the present inventors managed to locate 4 copies of sAD/GP1 complexes in the ASU. The model was manually fitted into electron density maps using Coot [Emsley et al. Acta crystallographica. Section D, Biological crystallography (2010) 66: 486-501] and refined using Phenix Refine [Adams et al., (2010) supra] in an iterative fashion.

Antibody Dependent Cellular-Mediated Cytotoxicity (ADCC) Assays

For measuring antibody dependent cellular-mediated cytotoxicity (ADCC) 293A cells were grown to confluency of 80-90% in 100 mm plate. Transfection mix of 1 ml containing 40 μg/ml of 25 kDa PEI (Polysciences) with 8 μg JUNV, MACV, or control plasmid in DMEM was made and incubated for 15 minutes at room temperature (RT). 2 ml media was removed from the culture, and 1 ml of the transfection mix was added to the plate. Cells were incubated with transfection mix 24 hours then lifted from the plate using 10 mM EDTA. The ability of Arenacept to promote ADCC was evaluated by measuring lactate dehydrogenase (LDH) release using LDH Cytotoxicity Detection kit (Roche Applied Science) according to the manufacturer's instructions. Target cells (T; 293A cells transfected with GPC of JUNV, MACV or irrelevant vector as control were incubated at 1×10⁵ cells/ml with or without 10 μg/ml Arenacept on ice for 1 hour. PBMCs were collected from human blood using CPT tubes, after extensive washes with PBS the cells were suspended in RPMI and plated in a 96-well round-bottom plate at different amounts. Subsequently, for each PBMCs-containing well 1×10⁴ target cells were added. 1% Triton X-100 was used as maximum release control and cells without PBMCs and no Arenacept as low spontaneous release controls. Plates were then incubated for 3 hours at 37° C., and supernatants were collected for LDH release determination. Percentage cytotoxicity was calculated as: (cells with Arenacept—cells without Arenacept)/(maximum release—spontaneous).

### Example 1

### Design of Soluble Apical Domain (sAD)

To develop a broadly reactive immunotherapy, the present inventors designed a TfR1-mimicry that would block the GP1 receptor binding sites. TfR1 is a large homodimeric type-II transmembrane glycoprotein (FIG. 1A) with a butterfly-like shape. Three subdomains make a single copy of the extracellular region of TfR1 (FIG. 1B): the helical domain that mediates dimerization, the protease-like domain, and the apical domain that is inserted between two β-strands of the protease-like domain (FIGS. 1B and 1C). The binding site for the TfR1-tropic Arenaviruses is the apical domain, which is not involved in the known physiological roles of TfR1 in binding transferrin or hereditary haemochromatosis protein. Thus, a soluble apical domain (sAD) was designed as a potential blocker of the TfR1-binding site. The design was based on the TfR1 gene from Neotoma Albigula (White-throated woodrat) (GenBank KF982058/UniProt A0A060BIS8) that can efficiently serve as an entry receptor for various clade-B & A/B Arenaviruses and has higher affinity to various GP1s compared with hTfR1. The present inventors eliminated a long loop (residues 301-326) that extends from the apical domain (FIGS. 1B and 1C), to mutate several hydrophobic residues that make part of the interface between the apical and the protease-like domains (FIGS. 1C and 1D) in order to make them hydrophilic, and to introduce two cysteine residues at the termini of this construct (FIG. 1C). This design should allow the expression of the apical domain as an isolated protein for making a receptor binding site competitor.

The designed sAD generated a soluble, folded, and stable protein. sAD was expressed fused to a His-tag at its C′ terminus using HEK293 cells in suspension. After affinity purification a defined monodisperse peak was obtained using size exclusion chromatography (FIG. 2A), indicating that sAD is a monomer in solution. Using circular dichroism spectroscopy, spectra was obtained for sAD that were characteristic to a folded protein, with a negative peak at a wavelength of 222 nm, indicating helical content (FIG. 2B). Following this negative peak at 222 nm, the thermal stability of sAD was monitored (FIG. 2C). The present inventors obtained a complex biphasic melting curve and thus did not attempt to fit a model to this data to derive a precise melting point. Nevertheless, sAD was completely stable up to 55° C. and the T_{M} was estimated to be approximately 65° C. Thus, the designed sAD yielded a monomeric, well-folded and stable protein when produced as isolated stand-alone domain.

### Example 2

### sAD Effectively Binds GP1 Domains of TfR1-Tropic Arenaviruses while Preserving a Native-Like Binding Mode

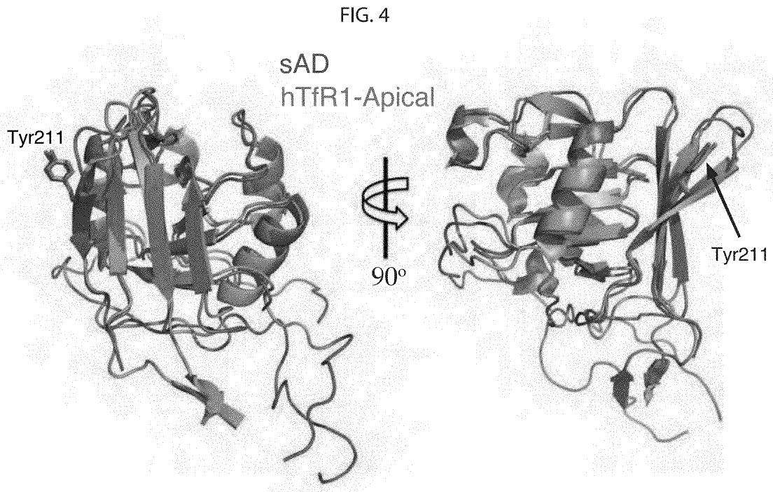

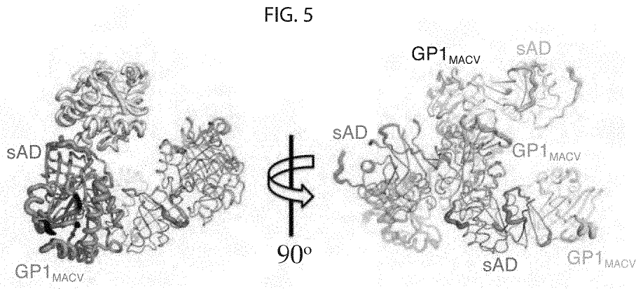

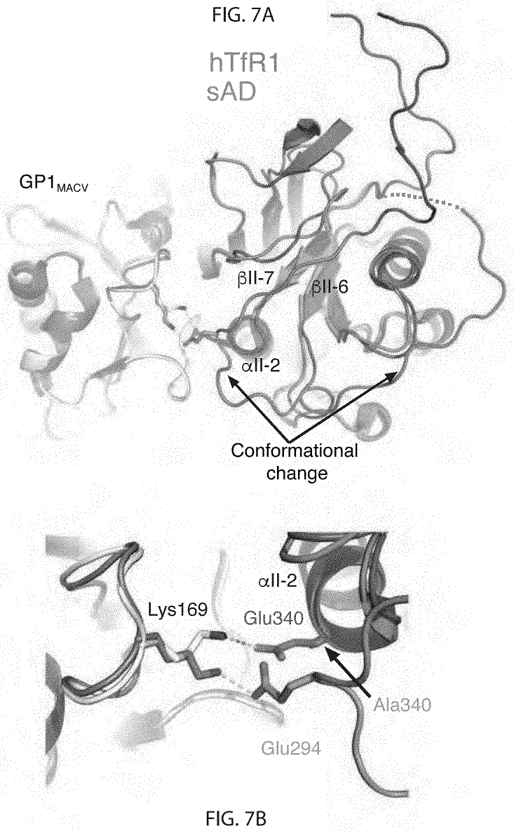

To evaluate whether sAD could target pathogenic TfR1-tropic viruses, a series of GP1 domains fused at their C′ termini to Fc-portions of antibodies were constructed. GP1 domains from JUNV, MACV, GTOV, and SABV, that are the major pathogenic Arenaviruses from clade-B, were included and further WWAV was included as a TfR1-tropic clade-A/B representative. Single cycle kinetics experiments were performed using surface plasmon resonance and the dissociation constants (KD) of sAD to the various representative GP1 domains were measured, in a configuration that allowed monovalent binding (FIGS. 8A-8E). All GP1 domains effectively bind sAD with KD values ranging from 4 nM for MACV to 1 μM for JUNV and WWAV (FIG. 2D). To verify the binding mode of sAD to GP1, the present inventors crystalized and solved the structure of GP1_{MACV} in complex with sAD to 2.7 Å resolution (Table 3 below). Crystals belonged to a tetragonal space group (P4322) with four copies of sAD/GP1_{MACV} in the asymmetric unit (FIG. 5). The designed sAD maintains the overall structure of hTfR1-apical domain (FIG. 4), and forms a complex with GP1_{MACV} (FIG. 2E) in a similar fashion to hTfR1. Out of two potential N-linked glycosylation sites of sAD (FIG. 1C), the present inventors observed density and hence modeled a glycan only at the Asn251 position (FIG. 2E). Most of the important interactions that GP1_{MACV} makes with hTfR1 were also formed with sAD, including the key interaction with Tyr211 (FIG. 2E). However, the present inventors did observe some structural differences; the long loop that connects parallel strands βII-6 & βII-7 of sAD that was mutated and partially eliminated (FIGS. 1B, 1C and 1D), changed its conformation (FIG. 7A). In the case of hTfR1, this loop contributes Glu294 that forms a salt-bridge with Lys169 of GP1_{MACV} (FIG. 7B). In the case of sAD Glu340 from αII-2 is substituting Glu294, and forms an equivalent salt-bridge with Lys169 (FIG. 7B). Overall, sAD mostly preserves the native structure of the apical domain from TfR1, and shows a remarkable broad-spectrum of reactivity against GP1s from clade-B and A/B NW Arenaviruses.

TABLE 3

| Data collection | |
|---|---|
| Wavelength (Å) | 0.9198 |
| Space group | P 4₃ 2 2 |
| Cell dimensions | |
| a, b, c (Å) | 104.6 104.6 281.4 |
| α, β, γ ° | 90 90 90 |
| Resolution (Å) | 50.00-2.7 (2.75-2.7) ^{a} |
| Rₚᵢₘ (%) | 3.8 (48.3) ^{a} |
| CC_{1/2} | 99.8 (30.0) ^{a} |
| I/σI | 17.2 (0.9) ^{a} |
| Completeness (%) | 94.3 (46.5) ^{a} |
| Multiplicity | 10.2 |
| Total reflections | 425370 |
| Unique reflections | 41703 |
| Refinement | |
| Resolution (Å) | 49.56-2.70 |
| No. of reflections | 36590 |
| R_{work}/R_{free} (%) | 24.1/27.4 |
| No. of atoms | |
| Protein | 9648 |
| Water | 51 |
| B factors | |
| Protein | 80.8 |
| Water | 69.6 |
| Ramachandran | |
| Favored (%) | 94.0 |
| Allowed (%) | 6.0 |
| Outlier (%) | 0.0 |
| Root mean square deviations | |
| Bond length (Å) | 0.03 |
| Bond angles ° | 0.746 |

^{a} Values in parentheses are for the highest resolution-shell

### Example 3

### Arenacept—an Immunoadhesin Based on sAD

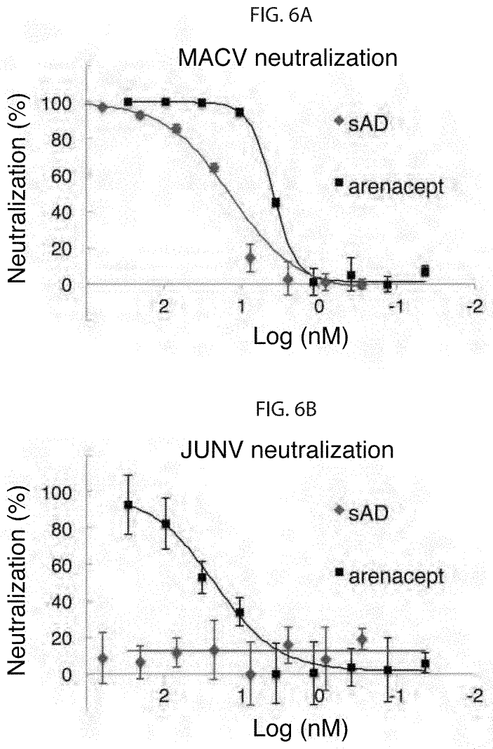

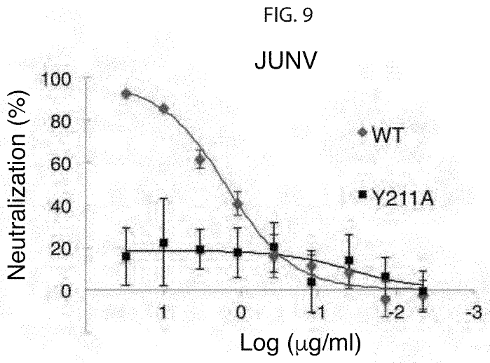

The present inventors constructed the sAD as an immunoadhesin by fusing to its C′-terminus an Fc portion of IgG1 in a configuration that enables avidity and named it “Arenacept”. First, the present inventors tested whether Arenacept can recognize the native spike complexes of the TfR1-tropic viruses. Using confocal fluorescence imaging it was demonstrated that Arenacept recognizes the native spike complexes of MACV, JUNV, GTOV, SABV and the clade-A/B WWAV when expressed in HEK293 cells (FIG. 3A). This recognition was specific, as the spike complex of the non TfR1-tropic Lassa Arenavirus was not recognized by Arenacept (FIG. 3A). Next, the present inventors examined whether Arenacept could neutralize pseudo-viruses bearing the spike complexes from the pathogenic clade-B viruses. MLV-based pseudo-viruses were generated that deliver luciferase when entering cells and were monitored for the reduction in infectivity in the presence of Arenacept (FIG. 3B). Applying Arenacept effectively neutralized MACV, JUNV, GTOV, and SABV with mean calculated IC50 values of 0.4-3.4 μg/ml (FIG. 3B). WWAV-pseudotyped viruses do not effectively infect HEK293 cells and hence neutralization could not be evaluated. Introducing Y211A mutation that eliminates critical contact with GP1 (FIG. 2E) into Arenacept resulted with abrogation of neutralization activity against JUNV (FIG. 9), indicating that Arenacept preserves the same binding mode observed for sAD (FIG. 2E). The similar IC50 values for the various viruses (FIG. 3B) compared with the mark difference in affinity of sAD to the GP1s (FIG. 2D) imply that avidity plays a role for Arenacept neutralization. Indeed, sAD has higher IC50 value for neutralizing MACV compared with Arenacept (FIGS. 6A-B). Thus, Arenacept successfully utilizes avidity and neutralizes all the four pathogenic clade-B viruses.

Arenacept can recruit the immune system to eliminate infected cells. Having an Fc portion as part of Arenacept may enable it to recruit the immune complement system and to induce antibody-dependent cellular cytotoxicity (ADCC). To test that, the present inventors transiently expressed the spike complexes of MACV and JUNV in HEK293 cells, applied peripheral blood mononuclear cells from healthy donors to the transfected HEK293 cells, and monitored cell-killing activity (FIG. 3C). A clear increase in cytotoxicity was observed as a function of the ratio between effector to target cells (FIG. 3C). Although Arenacept induced a stronger and more robust ADCC in the case of JUNV compared to MACV, in both cases ADCC activity was significant. Thus, Arenacept has the potential to promote clearing of infected cells on top of neutralizing viruses.

### Example 4

### Modified Form of Arenacept

The present inventors noted in the sAD (Arenacept) structure two potential N-linked glycosylation sites, one of them (on Asn204) was embedded within the site of interaction between the Arenacept and the different viral glycoproteins. Since glycosylation is stochastic, a portion of the produced Arenacept would have a glycan attached to this site and thus would prevent it from binding. Elimination of this glycosylation site could increase the amount of active molecules and hence enhance the neutralization potency of Arenacept.

Therefore serine 206 (part of the N—X—S glycosylation motif) was mutated to Alanine. The mutated version of Arenacept was expressed, purified and examined for neutralization properties. As evident from Table 4, below, and FIGS. 11A-D, Arenacept^{S206A} neutralizes pseudotyped TfR1-tropic arenaviruses and is more potent compared to the WT Arenacept discussed above.

TABLE 4
IC₅₀ values (μg/ml)

| | MACV | JUNV | SABV | GTOV |
|---|---|---|---|---|
| Arenacept | 0.5 | 2.4 | 3.5 | 1.8 |
| Arenacept^{S206A} | 0.3 | 1.4 | 2.0 | 1.5 |

### Example 5

### Arenacept In-Vivo Activity

Effectivity of Arenacept in-vivo is tested in murine models (e.g. guinea pig model of JUNV) as well as primate models to assess the severity of viral disease and survival in the presence or absence of Arenacept. For the guinea pig model of JUNV, viral disease is initiated by intramuscular administration of 1000 pfu JUNV. Next, Arenacept is administered intraperitoneally at different doses (e.g. 40 mg/kg) and at different time points (e.g. 2 and 6 days) after viral infection. Arenacept effectivity is assessed by measurement of viral load and percent survival post-infection as compared to non-treated animals.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition of matter comprising an isolated soluble polypeptide comprising an amino acid sequence of a Transferrin receptor protein 1 (TfR1) apical domain, said soluble polypeptide being capable of binding an Arenavirus.

2. The composition of matter of claim 1, wherein:
said amino acid sequence is devoid of a long loop;
said amino acid sequence comprises at least one deletion, insertion or point mutation that renders said TfR1 soluble;
said amino acid sequence of said TfR1 is as set forth in SEQ ID NO: 2, 4, 16 or 18;
said polypeptide comprises a stabilizing moiety; and/or
said polypeptide is of a length not exceeding 180 amino acid residues.

3. (canceled)

4. The composition of matter of claim 2, wherein said at least one point mutation:
comprises a substitution of a hydrophobic residue with a hydrophilic residue;
is at an interface between the apical domain and the protease-like domain of said TfR1; and/or
abolishes a glycosylation site of said TfR1.

5-6. (canceled)

7. The composition of matter of claim 64, wherein said glycosylation site comprises an N—X—S glycosylation motif.

8. The composition of matter of claim 7, wherein:
said Serine of said N—X—S glycosylation motif is mutated to any amino acid or mimetic thereof with the proviso that said amino acid is not Threonine;
said Serine of said N—X—S glycosylation motif is mutated to Alanine or mimetic thereof; and/or
said Asparagine of said N—X—S glycosylation motif is mutated to any amino acid or mimetic thereof with the proviso that said amino acid not Asparagine.

9-11. (canceled)

12. The composition of matter of claim 2, wherein said stabilizing moiety comprises a cysteine residue, and optionally wherein said cysteine residue comprises at least one cysteine residue at N- and/or C-termini of said polypeptide.

13-16. (canceled)

17. A composition of matter comprising a soluble polypeptide comprising an amino acid sequence of a TfR1 apical domain as set forth in SEQ ID NO: 6, said soluble polypeptide being capable of binding an Arenavirus.

18. The composition of matter of claim 1 wherein said polypeptide is attached to a heterologous moiety.

19. The composition of matter of claim 18, wherein said heterologous moiety is:
capable of inducing an antibody dependent cellular-mediated cytotoxicity (ADCC) response;
is for increasing avidity of the polypeptide;
is for multimerization; and/or
is a proteinaceous moiety.

20-22. (canceled)

23. The composition of matter of claim 19, wherein said proteinaceous moiety is selected from the group consisting of an immunoglobulin, a galactosidase, a glucuronidase, a glutathione-S-transferase (GST), a carboxy terminal peptide (CTP) from chorionic gonadotrophin (CGβ), and a chloramphenicol acetyltransferase (CAT).

24. (canceled)

25. The composition of matter of claim 23, wherein said immunoglobulin is an IgG Fc.

26. The composition of matter of claim 25, as set forth in SEQ ID NO: 8 or SEQ ID NO: 23.

27-28. (canceled)

29. A fusion protein comprising an amino acid sequence of a TfR1 apical domain and an amino acid sequence of IgG Fc, said fusion protein capable of binding an Arenavirus.

30. The fusion protein of claim 29, as set forth in SEQ ID NO: 8 or SEQ ID NO: 23.

31. (canceled)

32. The composition of matter of claim 1, being capable of neutralizing said Arenavirus.

33. (canceled)

34. A pharmaceutical composition comprising the composition of matter of claim 1, and a pharmaceutically acceptable carrier.

35. A method of treating or preventing an Arenavirus viral infection or disease associated therewith in a subject in need thereof, the method comprising administering to the subject a therapeutically effective amount of the composition of matter of claim 1, thereby treating or preventing the Arenavirus viral infection or disease associated therewith in the subject.

36. (canceled)

37. The method of claim 35, wherein said disease is a hemorrhagic fever.

38. (canceled)

39. An isolated polynucleotide encoding the polypeptide of claim 1.

40. The isolated polynucleotide of claim 39, comprising the nucleic acid sequence as set forth in SEQ ID NO: 1, 3, 5, 7, 15, 17 or 22.

41-43. (canceled)

44. A nucleic acid construct comprising the isolated polynucleotide of claim 39.

45. The nucleic acid construct of claim 44, further comprising a signal peptide.

46. A method of producing a polypeptide, the method comprising introducing the nucleic acid construct of claim 44 into a host cell; and culturing the host cell under conditions suitable for expressing the polypeptide.

47. (canceled)

48. A method of diagnosing an Arenavirus viral infection in a subject, the method comprising:
(a) contacting a biological sample from the subject with the fusion protein of claim 29, under conditions which allow the formation of immunocomplexes between an Arenavirus and said soluble polypeptide or said fusion protein; and
(b) determining a level of said immunocomplexes in said biological sample, wherein an increase in level of said immunocomplexes beyond a predetermined threshold with respect to a level of said immunocomplexes in a biological sample from a healthy individual is indicative of the Arenavirus viral infection.

49. The method of claim 48, further comprising corroborating the diagnosis using a diagnostic assay selected from antigen level measurement, antibody level measurement, virus isolation and/or genomic detection by reverse transcriptase-polymerase chain reaction (RT-PCR).

50. (canceled)


SEQUENCE LISTING (23 sequences)

SEQ ID NO: 1 - DNA, 2629 bp, homo sapiens
ctcgggacggaggacgcgctagtgttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcag

SEQ ID NO: 2 - protein, 760 aa, homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 3 - DNA, 2286 bp, neotoma albigula
atgatggatcaagccagatcagcaatctctaacttgtttggtggagaaccattgtcgtacacccggtttagcctggctcggcaagtagatggagataacagtcatgtggagatgaaattggctgtagatgaagaagaaactactgacaataacatgaaggccagtgtcagaaaacccagaagggttaatgggagactctgctatgggacaattgcagtagtcatctttttcttgattggatttatggtcggctacctgggctattgtaaacgcggagaacagaaagactgcgtgagactggcagacgcggagacagacacctcacaaagcttccaaatagaagaagacgttcctaaaccttctcgcttattttggtcagacctcaaaaatctattgtcagagaaactggatgctatagagttcactgacaccatcaagcagctgagccagaatacaaatacccctcgggaggctggatctcaaaaagatgaaaatcttgcctatgatattgaaaatcagttccatgactttaaactcagcaaagtctggcgagatgaacactatgtgaagatccaggtgaaaggcagcgttgctccaaactcggtgaccataacaaatgcaagtggtggcttgtacctagtggagtatcctgagggttatgtggcgtatagtaaagctacagaagttactggtaaactggtccatgctaattttggcactaaaaaggactttgaagatttagattatgctgtgaatggatctatagtgattgttagagcagggaaaatcactattgcagaaaaggttgcaaatgcccaaagctttaatgcaattggtgtcctgatatacatggacaggactaagttccccattgttagggcagacattccactctttggacatgctcatctaggaaccggtgacccatacacacctggctttccatctttcaaccatactcagtttccgccatcccaatcatcaggattgcctagtatacctgttcaaacaatctcaagagaggctgcagaaaagctatttcagaacatggaaagagactgtcctcgtagttggaacacagattcttcctgtaagctggaattgttacagaacagaaatgtgaagctcactgtgaacaatgcactgaaagaaacgagaatacttaacatctttggccttattaaaggttttgaggaaccagaccgttacattgtagtaggagcccagagagatgcctggggtcctggtgctgcaaagtccagtgtgggaacaggtcttctgttgaaacttgcccaagtattctcagatatggtttcaagaggtgggtttaaacccagcagaagcattatctttgccagctggagtgcaggagacttcggagctgttggtgccactgagtggctagagggatacctttcatctctgcatttaaaagctttcacttatattaatctggataaagttgtccttggtactagcaacttcaaagtttctgccagccctctattatatacacttattgggaagacaatgcaagatgtaaagcatccaattgatgggaaatctctatatcgagacagcaattggattagcaaagctgagaagctttcccttgacaatgctgctttcccttttcttgcatattctggaatcccagcagtttccttctgtttttgtgaggatgaggactatccttatttgggcactactttggatacctatgagagactgattgacaaagttcctcagctgaacaaaatggttcgtgcagcagcagaagtggctggtcagttcattattaaacttacccatgaaattgaactgaacctggactatgatatgtataacagcaaaatactggcatttgtgagggatctgaaccagttcagagcagatttcagggcgatgggtctgagtctacagtggctgtattctgctcgtggagacttcttccgtgctacatctagactaacgaatgatttccataatgctgagaaaacaaacagatttgtcatgagagaaatcaatgatcggattatgaaagtggaatatcacttcctgtcaccctacgtatctccaagagagtttcctttccgacacatcttctggggctctggctctcacactcttccagctttagtggagaacttgaaccttcgtcagagaaatattactgcttttaatgaaacactattcagaaaccagttggccctggctacttggactattcagggagttgcaaatgctctctctggtgacatttggaatattgacaatgagttttaa

SEQ ID NO: 4 - protein, 761 aa, neotoma albigula
MMDQARSAISNLFGGEPLSYTRFSLARQVDGDNSHVEMKLAVDEEETTDNNMKASVRKPRRVNGRLCYGTIAVVIFFLIGFMVGYLGYCKRGEQKDCVRLADAETDTSQSFQIEEDVPKPSRLFWSDLKNLLSEKLDAIEFTDTIKQLSQNTNTPREAGSQKDENLAYDIENQFHDFKLSKVWRDEHYVKIQVKGSVAPNSVTITNASGGLYLVEYPEGYVAYSKATEVTGKLVHANFGTKKDFEDLDYAVNGSIVIVRAGKITIAEKVANAQSFNAIGVLIYMDRTKFPIVRADIPLFGHAHLGTGDPYTPGFPSFNHTQFPPSQSSGLPSIPVQTISREAAEKLFQNMERDCPRSWNTDSSCKLELLQNRNVKLTVNNALKETRILNIFGLIKGFEEPDRYIVVGAQRDAWGPGAAKSSVGTGLLLKLAQVFSDMVSRGGFKPSRSIIFASWSAGDFGAVGATEWLEGYLSSLHLKAFTYINLDKVVLGTSNFKVSASPLLYTLIGKTMQDVKHPIDGKSLYRDSNWISKAEKLSLDNAAFPFLAYSGIPAVSFCFCEDEDYPYLGTTLDTYERLIDKVPQLNKMVRAAAEVAGQFIIKLTHEIELNLDYDMYNSKILAFVRDLNQFRADFRAMGLSLQWLYSARGDFFRATSRLTNDFHNAEKTNRFVMREINDRIMKVEYHFLSPYVSPREFPFRHIFWGSGSHTLPALVENLNLRQRNITAFNETLFRNQLALATWTIQGVANALSGDIWNIDNEF

SEQ ID NO: 5 - DNA, 516 bp, Artificial sequence
  Nucleic acid sequence of Tfr1 soluble apical domain
ggatccaagattcaggtcaaatgttcagctcctaactctgtcacaattactaacgcatcaggcggactgtatctggtggaataccccgagggctacgtggcctattctaaggccaccgaggtgacaggcaagctggtgcacgccaacttcggcaccaagaaggactttgaggacctggattacgccgtgaatggctccatcgtgatcgtgcgggccggcaagatcaccatcgccgagaaggtggccaacgcccagtccttcaatgccatcggcgtgctgatctacaaggacaggacaaagtatccaatctctagggcagatgagccactgcagggacactctggcctgccaagcatccctgtgcagaccatcagcagggaggcagcagagaagctgtttcagaacatggagcgggactgccccagatcctggaatacagatagctcctgtaagctggagctgctgcagaacaggaatgtgaagctgaccgtgaataactgcctgaaggagggtacc

SEQ ID NO: 6 - protein, 170 aa, Artificial sequence
  Amino acid sequence of Tfr1 soluble apical domain
GSKIQVKCSAPNSVTITNASGGLYLVEYPEGYVAYSKATEVTGKLVHANFGTKKDFEDLDYAVNGSIVIVRAGKITIAEKVANAQSFNAIGVLIYKDRTKYPISRADEPLQGHSGLPSIPVQTISREAAEKLFQNMERDCPRSWNTDSSCKLELLQNRNVKLTVNNCLKE

SEQ ID NO: 7 - DNA, 1230 bp, Artificial sequence
  Nucleic acid sequence of ARENACEPT
ggatccaagattcaggtcaaatgttcagctcctaactctgtcacaattactaacgcatcaggcggactgtatctggtggaataccccgagggctacgtggcctattctaaggccaccgaggtgacaggcaagctggtgcacgccaacttcggcaccaagaaggactttgaggacctggattacgccgtgaatggctccatcgtgatcgtgcgggccggcaagatcaccatcgccgagaaggtggccaacgcccagtccttcaatgccatcggcgtgctgatctacaaggacaggacaaagtatccaatctctagggcagatgagccactgcagggacactctggcctgccaagcatccctgtgcagaccatcagcagggaggcagcagagaagctgtttcagaacatggagcgggactgccccagatcctggaatacagatagctcctgtaagctggagctgctgcagaacaggaatgtgaagctgaccgtgaataactgcctgaaggagggtaccgagaacttgtacttccagggtagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatagcggccg

SEQ ID NO: 8 - protein, 407 aa, Artificial sequence
  Amino acid sequence of ARENACEPT
GSKIQVKCSAPNSVTITNASGGLYLVEYPEGYVAYSKATEVTGKLVHANFGTKKDFEDLDYAVNGSIVIVRAGKITIAEKVANAQSFNAIGVLIYKDRTKYPISRADEPLQGHSGLPSIPVQTISREAAEKLFQNMERDCPRSWNTDSSCKLELLQNRNVKLTVNNCLKEGTENLYFQGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 2237 bp, Neacomys spinosus
aacttgtttggtggagaaccattatcgtatactcggtttagcctggctcagcaagtagatggagataacagccatgtggagatgaaattggctgtagatgaagaggagaatgtggacaacatgaaggccagcgtcagaaaacccaaaaggtttaatggaggaagactctgctttgggacaattgcagtagtcatctttttcttgattggatttatgattggctacctgggatattgtaaacgcatagaacaaaaagaatgtgcgagactggcagaaacggagacagtcagttcacaaaacctccaaccagaagaagacattcctaaatcttctcgcttatattgggcagacctcaaaaaactgttgtcagagaaattggatgccatagacttcactgaccctatcaagcagctgagccaaaatacatacacccctcgggaggctggatctcaaaaagatgaaaatcttgcctattatattgaaaatcaatttcatgactttaaactcagcaaagtctggcgagatgagcattatgtgaagcttcaggtgaaaggcagcactgatcaaaactcagtgaccataataaattcaagtggtggcttgtaccttctggagagtcctgagggttatgtggcatatagcaaagctgcagaagttactggtaaactggtccatgctaattttggcaccaaaaaagactttgaaaacttaaattatgctgtgaatggatccttagtgattgttagagcaggggaaatcacttttgcagaaaaggttgcaaatgcccaaaactttaatgcaattggtgtcttgatatacatggacaagaataaattcccagttgttaaggcagaccttccactctttggacatgctcatctaggaactggtgacccatacacacctggctttccatctttcaaccatactcaatttccaccatctcagtcatcaggattgcctagtatacctgttcaaacaatctcaagagagactgcggacaagctatttcaaaatatggaaggaaactgtcctcctggttggaatgtagattcttcgtgtaagctagaattgacttccaataaaaatgtgaagctcactgtgaaaaatgaactgaaagagacaagaatacttaacatctttggagttattaaaggttttgaggaaccaggccattacattgtagtaggagcccagagagattcctggggtcctggtgctgcaaagtccagtgtgggaacagctcttctgttgaaacttgcccaaacattctcagatatggttttaagaggtggatttaaacccagcagaagcattatctttgccagctggactgcaggagactttggagctgttggtgccaccgagtggctagagggatacctttcatctctgcatttaaaagccttcacttacattaatctggataaagttgtccttggttctagcaacttcaaggtttctgccagccctctgttatatacacttattgagaagacaatgcaggacgtaaagcatccaattgatgggaagtctctatatcgagatagcaactggatcagcaaagttaagaaactttcccttgacaacgccgctttcccgtttcttgcatattctggaatcccagcagtttctttctgtttttgtgaggatgaggactatccttatttggacactactttggatacctatgagttactaattcagaaagttcctcagttgggtaaaatggttcgtgtagctgcagaagtggctggtcagttcattattaaacttactcatgaaattgaattgaacctggactatgacatgtataacaacaaatactgtaacaacaaaatactgtcatttgtgagggatctgaaccagttcagagcggatatcagggagatgggtctaagtctacagtggctgtattctgctcgcggagactactttcgtgctacatctagactaacatctgacttccataatgcagagaaaacaaacagatttgtcatgagggaaatcaatgaccgtattatgaaagtggaatatcatttcctgtcaccctatgtatctcccagagagtctcctttccgacacatcttctggggttatggctctcacactctcccagctttaatggagaacttgaagcttcggcagaaaaatattagtgcttttaatgaaacactcttcagaaaccagttggccctagctacttggactattcagggagttgcaaatgccctctc

SEQ ID NO: 10 - protein, 746 aa, Neacomys spinosus
NLFGGEPLSYTRFSLAQQVDGDNSHVEMKLAVDEEENVDNMKASVRKPKRFNGGRLCFGTIAVVIFFLIGFMIGYLGYCKRIEQKECARLAETETVSSQNLQPEEDIPKSSRLYWADLKKLLSEKLDAIDFTDPIKQLSQNTYTPREAGSQKDENLAYYIENQFHDFKLSKVWRDEHYVKLQVKGSTDQNSVTIINSSGGLYLLESPEGYVAYSKAAEVTGKLVHANFGTKKDFENLNYAVNGSLVIVRAGEITFAEKVANAQNFNAIGVLIYMDKNKFPVVKADLPLFGHAHLGTGDPYTPGFPSFNHTQFPPSQSSGLPSIPVQTISRETADKLFQNMEGNCPPGWNVDSSCKLELTSNKNVKLTVKNELKETRILNIFGVIKGFEEPGHYIVVGAQRDSWGPGAAKSSVGTALLLKLAQTFSDMVLRGGFKPSRSIIFASWTAGDFGAVGATEWLEGYLSSLHLKAFTYINLDKVVLGSSNFKVSASPLLYTLIEKTMQDVKHPIDGKSLYRDSNWISKVKKLSLDNAAFPFLAYSGIPAVSFCFCEDEDYPYLDTTLDTYELLIQKVPQLGKMVRVAAEVAGQFIIKLTHEIELNLDYDMYNNKYCNNKILSFVRDLNQFRADIREMGLSLQWLYSARGDYFRATSRLTSDFHNAEKTNRFVMREINDRIMKVEYHFLSPYVSPRESPFRHIFWGYGSHTLPALMENLKLRQKNISAFNETLFRNQLALATWTIQGVANALS

SEQ ID NO: 11 - DNA, 2282 bp, Artibeus jamaicensis
  misc_feature (53)..(53): n is a, c, g, or t
aacttgtttagtggagaaccattgtcatacacccggtttagtctcgcacggcnagtggatggcgataacagtcgtgtggagatgaaactagcagcagacgaggaagaaaatgatgacaatcacatgaggagcaattatgcccatgttgcaaaatcaaaaaagtttaatggaagtatctgctacggaactattgctataatcatctttttcttcattggatttatgattggctacttgggctattgtaaacgtgtggaaccaaaagctgaatgtgaaagaacagagtctccgggtacagaggagtctccagtggagacagtagactacctttccgaaacacctccccgtttgttttggtcacacctcaaagcagtgttgtcagagaaactgaataacgttgacttcgccaacacaatcaagcagctaaatgaaaattcctatgtccctcgtgaggctggatctcaacaagatgaaagccttgctttttatcttgaaggtcaatttcgtaaatttcaactcagcaaagcctggcacgatgaacattttgtcaaggttcaggtcaaaggcagtgctcaaaatgcagtgaccatagtggctgtgagcagtggtgcaggatacctggtggagaaccctgcaggttacgtggcgtacagcaagaccggaacagttactggtaaactggtccatgctaattttggcactaaacaagactttgaaagttcaagcattcctgtgaatggatctttagtgattgttagagcagggaaaatcacttttgctgaaaaggttgcaaatgctcaaagttttaatgcaattggtgttttgatctacatggactatactaaatttcccattgttaatgcacagcttccactctttggacatgctcatctgggaacaggtgacccttacacacccggatttccttctttcaatcacactcagtttccaccatcgcagtcatcaggattgcctaccatacctgtccaaacaatttccagagccgctgcagagaaactgtttgaaaatatggagcaactctgtccttctgcttggagaacagactcttcatgtaggctggtaacaaaaaaggataggaatgtgaaactcactgtgagcaatgtactgaaagagacaagaattttaaatgtctttggtgttattaaaggctttgaagaacccgatcgctatgttgtggtgggggcccagagggatgcctggggccctggagctgtgaagtccagtgtgggaacagctctcctattggaacttgcccggatattttctgatatggtcttaaaaggtgggtttaagcccagcagaagcattgtcttcgccagctggagcgcaggagactttggagccgtgggcgctactgaatggctggagggatacctttcctccttgcatttaaaggctttcacttacattaatctggataaagctgtccttagtgacggcaacttaaaggtttctgccagcccactgttgtattcacttattgagaaaacgatgcaagaagtgaaacatccagttactgggctttctttatatcgggatagcaactggatcaaaaaagttgagaaactttctttggataatgctgctttccctttccttgcgtattctggaatcccagcagtttctttctgtttttgtgaggacttggattatccttatttaggtactaccctggatacctatgaggcactgtatgggagagtccctgagttgaacagaatggtacgtggagcagcagaagtggctggccagctcatgattagacttacccatgatgttgaattgaacctgaactatgacatgtataatgaggaaatacttgaatttgtgagggatatgaaccagttcagaagagacgtaaaggagatgggtctgatctacagtgctgtatctgctcgcggagacttcttcgtgctacttctaactcacagcagatatagatgcgaaagacaacgattgtcacgtgctacttctaaactcacagcagattataagaatgcggagagaaccaacagatttgtcatgagggaaatcaatgaccgtatcatgagagtggaataccacttcctctcaccctatgtatctccaagagattctcctttccgacatatcttctggggctctggctctcacactctgtcagctttactagagcacttaaagctgcgtcgagaaaataacagtgctttcaatgaaacactgttgagaaaccagttggctttagcaacttggacaattcagggagctgcaaatgctctctc

SEQ ID NO: 12 - protein, 761 aa, Artibeus jamaicensis
  misc_feature (18)..(18): Xaa can be any naturally occurring amino acid
NLFSGEPLSYTRFSLARXVDGDNSRVEMKLAADEEENDDNHMRSNYAHVAKSKKFNGSICYGTIAIIIFFFIGFMIGYLGYCKRVEPKAECERTESPGTEESPVETVDYLSETPPRLFWSHLKAVLSEKLNNVDFANTIKQLNENSYVPREAGSQQDESLAFYLEGQFRKFQLSKAWHDEHFVKVQVKGSAQNAVTIVAVSSGAGYLVENPAGYVAYSKTGTVTGKLVHANFGTKQDFESSSIPVNGSLVIVRAGKITFAEKVANAQSFNAIGVLIYMDYTKFPIVNAQLPLFGHAHLGTGDPYTPGFPSFNHTQFPPSQSSGLPTIPVQTISRAAAEKLFENMEQLCPSAWRTDSSCRLVTKKDRNVKLTVSNVLKETRILNVFGVIKGFEEPDRYVVVGAQRDAWGPGAVKSSVGTALLLELARIFSDMVLKGGFKPSRSIVFASWSAGDFGAVGATEWLEGYLSSLHLKAFTYINLDKAVLSDGNLKVSASPLLYSLIEKTMQEVKHPVTGLSLYRDSNWIKKVEKLSLDNAAFPFLAYSGIPAVSFCFCEDLDYPYLGTTLDTYEALYGRVPELNRMVRGAAEVAGQLMIRLTHDVELNLNYDMYNEEILEFVRDMNQFRRDVKEMGLIYSAVSARGDFFVLLLTHSRYRCERQRLSRATSKLTADYKNAERTNRFVMREINDRIMRVEYHFLSPYVSPRDSPFRHIFWGSGSHTLSALLEHLKLRRENNSAFNETLLRNQLALATWTIQGAANALS

SEQ ID NO: 13 - DNA, 2283 bp, Calomys callosus
atgatggatcaagccagatcagcaatctctaacttgtttggtggagaaccattatcatatactcgatttagcctggctcggcaagtagatggagataatagccatgtggagatgaaattagctgtagatgaagaagaaaatgtggacaacatgaaggccagcgtcagaaaacccagaaggtttaatgggagactctgctttgggacaattgcagtagtcatctttttcttgattggatttatgataggctatctgggatattgtaaacgcatagaacaaaaagactgtgcaagaccggcagaaatggaaacaatcaattcacaaaatctccaaccagaagaagacattcctaaatcttctcgcttatattgggcagacctcaaaaagctgttgtcagagaaactggatgccatagacttcactgacaccatcaagcagctgagccaaaatacgtatacccctcgggaggctggatctcaaaaagatgaaaatcttgcctactatattgaaaatcaatttcatgacttaaaactcagcaaagtttggcgagatgaacattatgtgaagcttcaggtgaaaagcagcgttgctcaaaactcagtgaccataataaatgcaagtaatggcgtatacctattggagagtcctgcgggttatgtggcatatagcaaagctgcagaagttactggtaaactggtccatgctaattttggcaccaaaaaagactttgaaaacttaaattatgctgtgaatggatccttagtgattgttagagcaggagaaattacttttgcagaaaaggttgcaaatgcccaaagccttaatgcaattggtgtcttgatatacatggacaagaataaattcccagttgttaaggcagacctttcactctttggacatgctcatctaggaactggtgacccatacacacctggctttccatctttcaaccatactcaatttccaccatctcagtcatcaggattgcctagtatacctgttcaaacaatctcaagagaggctgcggacaacctatttcaaaacatggagggaaactgtcctcctagttggaacgtagattcttcatgtaagctagagttgacttcagataaaactgtgaagctcgttgtgagaaatgaactgaaagagacaagaatacttaacatctttggagttattaaaggttttgaggaaccagatcgttacattgtagtaggagcccagagagattcctggggtcctggtgctgcaaagtccagtgtgggaacagctcttctgttgaaacttgcccagacattctcagatatggttttaagaggtggatttaaacccagcagaagcattatctttgccagctggactgcaggagactttggagctgttggtgccacggagtggctagagggatacctttcatctctgcatttaaaagccttcacttacattaatctggataaagttgtccttggtaccagcaaattcaaggtttctgccagtcctctgttatatacacttattgagaagacaatgcaggacgtaaagcatccaattgatgggaaatctctgtatcgagacagcaactggatcagcaatgttaagaaactttcctttgacaacgccgctttcccttttcttgcatattctggaatcccagcagtttctttcagtttttgtgaggatgaagactatccttatttggacacgattttggatacctatgacctattgattcagaaagttcctcagctgaacaaaatggttcgtgtagctgcagaagtggctggtcagttcattattaaacttactcatgaaattgaactgaacctggactacgatatgtataacaacaaaatactgtcatttgtgagggatctgaaccagttcagagcggatatcagggagatgggtctaagtctacagtggctgtattctgctcgtggagactactttcgtgctacatctagactaacatctgacttccataatgcggagaaaacaaacagatttgtcatgagggaaatcaatgaccgtattatgaaagtggaataccacttcctgtcaccttatgtatctcccagagagtctcctttccgacacatcttctggggttatggttctcacacactctcagctttaatggagaacttgaagcttcggcagacaaatattagtgcttttaatgaaacgctcttcagaaaccagttggccctagcttcttggactatacagggagttgcaaatgccctctctggtgacatttggaatattgacaatgagttttaa

SEQ ID NO: 14 - protein, 760 aa, Calomys callosus
MMDQARSAISNLFGGEPLSYTRFSLARQVDGDNSHVEMKLAVDEEENVDNMKASVRKPRRFNGRLCFGTIAVVIFFLIGFMIGYLGYCKRIEQKDCARPAEMETINSQNLQPEEDIPKSSRLYWADLKKLLSEKLDAIDFTDTIKQLSQNTYTPREAGSQKDENLAYYIENQFHDLKLSKVWRDEHYVKLQVKSSVAQNSVTIINASNGVYLLESPAGYVAYSKAAEVTGKLVHANFGTKKDFENLNYAVNGSLVIVRAGEITFAEKVANAQSLNAIGVLIYMDKNKFPVVKADLSLFGHAHLGTGDPYTPGFPSFNHTQFPPSQSSGLPSIPVQTISREAADNLFQNMEGNCPPSWNVDSSCKLELTSDKTVKLVVRNELKETRILNIFGVIKGFEEPDRYIVVGAQRDSWGPGAAKSSVGTALLLKLAQTFSDMVLRGGFKPSRSIIFASWTAGDFGAVGATEWLEGYLSSLHLKAFTYINLDKVVLGTSKFKVSASPLLYTLIEKTMQDVKHPIDGKSLYRDSNWISNVKKLSFDNAAFPFLAYSGIPAVSFSFCEDEDYPYLDTILDTYDLLIQKVPQLNKMVRVAAEVAGQFIIKLTHEIELNLDYDMYNNKILSFVRDLNQFRADIREMGLSLQWLYSARGDYFRATSRLTSDFHNAEKTNRFVMREINDRIMKVEYHFLSPYVSPRESPFRHIFWGYGSHTLSALMENLKLRQTNISAFNETLFRNQLALASWTIQGVANALSGDIWNIDNEF

SEQ ID NO: 15 - protein, 760 aa, homo sapiens; identical to SEQ ID NO: 14

SEQ ID NO: 16 - DNA, 587 bp, homo sapiens
aagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagat

SEQ ID NO: 17 - protein, 195 aa, neotoma albigula
KIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 18 - DNA, 587 bp, neotoma albigula
aagatccaggtgaaaggcagcgttgctccaaactcggtgaccataacaaatgcaagtggtggcttgtacctagtggagtatcctgagggttatgtggcgtatagtaaagctacagaagttactggtaaactggtccatgctaattttggcactaaaaaggactttgaagatttagattatgctgtgaatggatctatagtgattgttagagcagggaaaatcactattgcagaaaaggttgcaaatgcccaaagctttaatgcaattggtgtcctgatatacatggacaggactaagttccccattgttagggcagacattccactctttggacatgctcatctaggaaccggtgacccatacacacctggctttccatctttcaaccatactcagtttccgccatcccaatcatcaggattgcctagtatacctgttcaaacaatctcaagagaggctgcagaaaagctatttcagaacatggaaagagactgtcctcgtagttggaacacagattcttcctgtaagctggaattgttacagaacagaaatgtgaagctcactgtgaacaatgcactgaaagaaac

SEQ ID NO: 19 - protein, 19 aa, Artificial sequence
  Amino Acid sequence of signal peptide for expression of sAD and arenacept
GLCCVLLLCGAVFVSPAGA

SEQ ID NO: 20 - DNA, 2277 bp, Sigmodon hispidus
atgatggatcaagccagatcagcaatctctaacttgtttggtggagaaccattatcatatacccggtttagcctggctcggcaagcagatggagataacagccatgtggagatgaaattggctgtagatgaagaagaaaatatggacaacatgaaggctagtgtcagaaaacccagaaaatttaatggaagactctgctttgggacagttgcagtagtcatctttttcttaattggatttatgactggctacctggggtactgtaaacgcatagaacaaaaagactgtgtgaaagtggcagaagcggagacagtcaattcacagaacttccaaacagaagaagacattcctaaatcttctcgcttgtattgggcagacctcaaaaaactgttgtcagagaaactgaacaccatagagttcacagacaccatcaagcagctgagccagaatacccctcgggaggctggatctcaaaaagatgaaaatcttgcctattatattgaaaatcagtttcaagactttaaactcagcaaagtctggcgagatgaacattatgtgaagatccaggtgaaaggcagcaatgctcaaaactcggtgaccataataaatgataatggtggcttgtacctgttgaagaatcttacaggttatgtggcatatagcaaagctgcagaagttactggtaaactggtccatgctaattttggcactaaaaaagactttgaaaacttaaattatgctgtgaatggatccttagtgattgttagagcaggggaaatcacttttgcagaaaaggttgcaaatgcacaaagctttaatgcaattggtgtcttgatatacatggacaagaataaattcccagttgttaaggcagaccttgcacgttttggacatgctcatctaggaactggtgacccatacacacctggctttccatctttcaatcatactcagtttccgccatctcagtcatcgggattgcctaggatacctgttcaaacaatttcaagagaggctgctgacaagctatttcaaaacatggaaggaaagtgcccttctagttggaacgtagattcttcatgtaaactggaattgacatcagataaaaatgtgaagctcagtgtgaacaacgagttgaaagagacaagaatacttaatatctttggcgttattaaaggttttgaggaaccagaccgttacgttgtagtaggagcccagagagattcctggggtcctggtgctgcaaagtccagtgtgggaacagctcttctgttgaaacttgcccaaacattttcagatatggttttaagaggtggatttaaacccattaggagcattatctttgccagctggactgcaggagacttcggagctgttggtgccaccgagtggctagagggatacctttcatctctgcacataaaagccttcacttacattaatctggataaagttgtccttggtactaacaacttcaaagtttctgctagccctctattgtatacacttattgagaagacaatgcaggatgtaaagcatccaattgatgggagatctctatatcgagacagcaactggatcaacaaagttgagaaactttcccttgacaatgccgccttcccttttctggcatattctggaatcccagcagtttctttctgtttttgtgaggatgaggattatccttatttggatactatgctggatacgtatgagttactaattcagaaagttcctcagctggacaaaatggttcgtgtagctgcagaagtggctggtcagttcattattaaacttactcatgaaattgaactgaacctggactatgatatgtataacaacaaaatactgtcatttgtgagggatctgaaccagttccgagcagatatcagggacatgggtctaagtctacagtggctgtattctgctcgcggagactactttcgtgctacatctagactaacatctgacttccataatgcggagaaaacaaacagatttgtcatgaggcaaatcaatgaccgtattatgaaagtagaatatcacttcctgtcaccctatgtatctcccagagagtctccttttcgacacatcttctggggttatggctctcacactctttcagctttaatggagagtttgaagcttcgacagaaaaatattagcgcttttaatgaaacactcttcagaaaccagttggccttagcttcgtggactattcaaggagttgcaaatgccctctctggtgacatttggaatattgacaatgagttttaa

SEQ ID NO: 21 - protein, 758 aa, Sigmodon hispidus
MMDQARSAISNLFGGEPLSYTRFSLARQADGDNSHVEMKLAVDEEENMDNMKASVRKPRKFNGRLCFGTVAVVIFFLIGFMTGYLGYCKRIEQKDCVKVAEAETVNSQNFQTEEDIPKSSRLYWADLKKLLSEKLNTIEFTDTIKQLSQNTPREAGSQKDENLAYYIENQFQDFKLSKVWRDEHYVKIQVKGSNAQNSVTIINDNGGLYLLKNLTGYVAYSKAAEVTGKLVHANFGTKKDFENLNYAVNGSLVIVRAGEITFAEKVANAQSFNAIGVLIYMDKNKFPVVKADLARFGHAHLGTGDPYTPGFPSFNHTQFPPSQSSGLPRIPVQTISREAADKLFQNMEGKCPSSWNVDSSCKLELTSDKNVKLSVNNELKETRILNIFGVIKGFEEPDRYVVVGAQRDSWGPGAAKSSVGTALLLKLAQTFSDMVLRGGFKPIRSIIFASWTAGDFGAVGATEWLEGYLSSLHIKAFTYINLDKVVLGTNNFKVSASPLLYTLIEKTMQDVKHPIDGRSLYRDSNWINKVEKLSLDNAAFPFLAYSGIPAVSFCFCEDEDYPYLDTMLDTYELLIQKVPQLDKMVRVAAEVAGQFIIKLTHEIELNLDYDMYNNKILSFVRDLNQFRADIRDMGLSLQWLYSARGDYFRATSRLTSDFHNAEKTNRFVMRQINDRIMKVEYHFLSPYVSPRESPFRHIFWGYGSHTLSALMESLKLRQKNISAFNETLFRNQLALASWTIQGVANALSGDIWNIDNEF

SEQ ID NO: 22 - DNA, 1053 bp, Artificial Sequence
  Nucleic acid sequence of  S206A ARENACEPT
ggatccaagattcaggtcaaatgttcagctcctaactctgtcacaattactaacgcagcaggcggactgtatctggtggaataccccgagggctacgtggcctattctaaggccaccgaggtgacaggcaagctggtgcacgccaacttcggcaccaagaaggactttgaggacctggattacgccgtgaatggctccatcgtgatcgtgcgggccggcaagatcaccatcgccgagaaggtggccaacgcccagtccttcaatgccatcggcgtgctgatctacaaggacaggacaaagtatccaatctctagggcagatgagccactgcagggacactctggcctgccaagcatccctgtgcagaccatcagcagggaggcagcagagaagctgtttcagaacatggagcgggactgccccagatcctggaatacagatagctcctgtaagctggagctgctgcagaacaggaatgtgaagctgaccgtgaataactgcctgaaggagggtaccgagaacttgtacttccagggtagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactac

SEQ ID NO: 23 - protein, 407 aa, Artificial Sequence
  Amino acid sequence of  S206A ARENACEPT
GSKIQVKCSAPNSVTITNAAGGLYLVEYPEGYVAYSKATEVTGKLVHANFGTKKDFEDLDYAVNGSIVIVRAGKITIAEKVANAQSFNAIGVLIYKDRTKYPISRADEPLQGHSGLPSIPVQTISREAAEKLFQNMERDCPRSWNTDSSCKLELLQNRNVKLTVNNCLKEGTENLYFQGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK